(19) United States
(12) Patent Application Publication
(10) Pub. No.: US 2021/0324477 A1
(43) Pub. Date: Oct. 21, 2021

(54) GENERATING CANCER DETECTION PANELS ACCORDING TO A PERFORMANCE METRIC

(71) Applicant: Grail, Inc., Menlo Park, CA (US)

(72) Inventors: Jing Xiang, Menlo Park, CA (US); Anton Valouev, Palo Alto, CA (US)

(21) Appl. No.: 17/233,548

(22) Filed: Apr. 19, 2021

Related U.S. Application Data
(60) Provisional application No. 63/013,512, filed on Apr. 21, 2020.

(51) Int. Cl. C12Q 1/6886; G16B 40/00

(52) U.S. Cl. CPC C12Q 1/6886; G16B 40/00; C12Q 2600/106; C12Q 2600/118; C12Q 2600/156

(57) ABSTRACT

A system generates a cancer detection panel. The system is configured to generate an assay having a minimized size and number of genomic regions while still detecting the presence of cancer at or above a specific performance threshold. To select the genomic regions for the panel, the system employs a classification model. The classification model receives a set of genomic regions that may be associated with disease presence. The model then determines a sensitivity score for each genomic region and ranks the regions according to their score. The sensitivity score is based on a likelihood that variations in the genomic region are indicative of cancer. The model then selects genomic regions for the panel based on their rank. The model only selects as many genomic indicators as are needed for desired detection performance. The genomic regions can be associated with solid or liquid cancers, viral regions, or cancer hotspots.

### CROSS REFERENCE TO RELATED APPLICATIONS

The application claims the benefit of Provisional Application No. 63/013,512, filed on Apr. 21, 2020, the contents of which are incorporated herein by reference.

### FIELD OF ART

This disclosure relates to generating a disease detection panel, and, more specifically, to generating a cancer detection panel using a detection capability model.

### DESCRIPTION OF THE RELATED ART

Computational techniques can be used on DNA sequencing data to identify mutations or variants in DNA that can correspond to various types of cancer or other diseases. However, designing disease detection panels that efficiently pull-down sequencing data for identification of variants and mutations is a challenging process. Typically, disease detection panels include a large number of genomic regions selected for the panel. The included regions are selected because a variation in those regions have been previously shown to indicate a disease presence and/or a disease type. However, oftentimes, the included regions are not curated in any manner and the resulting panel is large and costly.

### SUMMARY

Disclosed herein is a method for generating a reduced gene panel for disease classification. The method may be implemented by a computer system. To begin, the system obtains sequencing data for a first set of genomic regions. For example, a set of 50 genomic regions. The system derives a plurality of feature values from the sequencing data for the first set of genomic regions.

The system then applies a classification model to the feature values. The classification model predicts a disease classification using the feature values. To do so, the classification model generates a set of model coefficients corresponding to the first set of genomic regions. The system then ranks the genomic regions according to their model coefficients. For example, the genomic region with the highest model coefficient is ranked first.

The system identifies a first subset of the genomic regions that optimizes the disease classification based on the rankings. For example, by selecting the 41 genomic indicators from the first set of genomic indicators having the highest model coefficients. In turn, the system generates a reduced gene panel comprising the first subset of genomic regions, e.g., a gene panel including the 41 genomic indicators in the subset.

In some embodiments, the sequencing data is obtained from sequencing cell-free nucleic acid molecules existing in biological samples obtained from a plurality of patients. In this way, the first set of genomic regions can include at least one of cancer-related genes, mutation hotspots, and/or viral regions. In some examples, the first set of genomic regions comprises genomic regions associated with a high signal cancer or a liquid cancer.

In some embodiments, the feature values comprise a maximum allele frequency of a variant at each genomic region in the first set of genomic regions. In various examples, the features values can represent features corresponding to at least one of a presence or absence of a variant, a mean allele frequency, a total number of small variants, and an allele frequency of true variants. A variant can be a single nucleotide variant, an insertion, and/or a deletion.

In some embodiments, the classification model comprises a logistic regression model. Thus, the set of model coefficients comprises regression coefficients obtained by training the logistic regression model with the derived feature values.

As described above, the system identifies a first subset of the genomic regions that optimize the disease classification. In some embodiments, to identify the first subset, the system, at an initial iteration, trains the classification model to predict a disease classification based on the feature values corresponding to the first genomic region. That is, a first genomic region corresponds to the highest ranked genomic region. The system then determines a performance metric of the classification model trained on the first genomic region.

To continue, at subsequent iterations, the system retrains the classification model by incorporating the remaining ranked genomic regions and evaluating the performance metric after each additional genomic region is incorporated. The system, with each subsequent iteration, applies a greedy algorithm to add a next-highest-ranked genomic region of the remaining ranked genomic regions to the classification model. Thus, the system retrains the classification model using feature values associated with the added next-highest-ranked genomic region and previously added genomic regions from preceding iterations. Accordingly, the system then determines a performance metric for the retrained classification model, and evaluates the performance metrics obtained for each iteration. Based on the evaluated performance metrics, the system identifies to identify the first subset of genomic regions that yields an optimized performance metric.

In some embodiments, the optimized performance metric is a maximum performance metric achieved by the classification model. For example, the optimized performance metric can be an optimized sensitivity level at a predetermined specificity level for a set of genomic indicators. The performance metric obtained with the reduced gene panel is substantially similar to a performance metric obtained with a full gene panel comprising the full first set of genomic regions.

In some embodiments, the first set of genomic regions comprises genomic regions associated with high signal cancers and has a set size of approximately 2 Mb. Thus, the first subset of genomic regions can have a subset size of less than 300 kb but could be other sizes. Accordingly, the reduced gene panel comprises a total panel size not exceeding 300 kb.

In some cases, the system may determine a second subset of genomic regions using a second set of genomic regions. In this case, the system identifies a second subset of genomic regions that further improves the disease classification achieved by the first subset of genomic regions. Once identified, the system generates the reduced gene panel comprising the first subset of genomic regions and the second subset of genomic regions.

To accomplish this, the system obtains a second set of sequencing data for a second set of genomic regions. The system then tanks the second set of genomic regions and identifies the second subset of genomic regions based on the ranked second set of genomic regions. In an example, the second set of genomic regions may be ranked according to the frequency of somatic mutations per patient, and/or the frequency normalized by a coding region length.

In some embodiments, other additional subsets of genomic regions using additional set of genomic regions. For example, the system identifies a third subset of genomic regions that further improves the disease classification achieved by the reduced gene panel. The system then includes the third subset of genomic regions in the reduced gene panel. The third subset of genomic regions can optimize a disease-type prediction accuracy of the reduced panel. Further, the third set of genomic regions can be cancer-specific genes and hotspots.

Some additional genomic regions that may be included include hotspot regions corresponding to single nucleotide variants, insertions, or deletions. Another genomic region can include viral target regions correspond to viral-associated cancers. In these cases, the classification model may select any number of the genomic regions to include in the reduced panel.

In some embodiments, the disease classification may comprise a binary classification for predicting cancer or non-cancer. The classification may also comprise and/or a multi-class classification for predicting a cancer type.

In some embodiments, the system may be implemented on a non-transitory computer-readable medium storing one or more programs. The programs can include instructions which, when executed by an electronic device including a processor, cause the device to perform any of the methods of the preceding claims.

In some embodiments, the electronic device may comprise one or more processor, memory, and one or more programs. The one or more programs can be stored in the memory and configured to be executed by one or more processors of the device. The one or more programs including instructions for performing any of the methods of the preceding claims.

As described above, the system can generate a disease detection (e.g., cancer) assay panel. To generate the panel, the system can select genomic regions from any of (i) a first set of genomic regions associated with high signal cancer genes and liquid cancer genes, (ii) a second set of genomic regions associated with cancer-specific genes and cancer-specific hotspot, and (iii) a third set of genomic regions associated with hotspots for single nucleotide variants or indels, and (iv) a fourth set of genomic regions associated with viral targets. The system then generates the cancer assay panel comprising a plurality of probe sets. Each probe set in the plurality of probe sets can comprise a pair of probes for targeting at least one of the genomic regions in the first, second, third, and fourth sets of genomic regions.

In selecting the genomic regions from the first, second, third, and/or fourth sets of genomic regions, the system may apply a classification model to assess a contribution of each genomic region to a detection sensitivity of the cancer assay panel.

In some embodiments, the first set of genomic regions comprises one or more genomic regions disclosed in Table 1 herein; the third set of genomic regions comprises one or more genomic regions disclosed in Table 3, Table 4, Table 5, and/or or Table 6 herein. In some embodiments, the system selects a fifth set of genomic regions that improves the detection sensitivity of the panel, and the fifth set of genomic regions comprises one or more genomic regions disclosed in Table 2 herein.

In some embodiments, the second set of genomic regions comprises one or more of CASP8, IDH1, TERT1, and EGFR. In some embodiments, the fourth set of genomic regions comprises one or more sites located at one or more genomic regions in HPV16, HPV18, EBV, and HBV.

The system may generate a panel using the genomic regions indicated herein. The panel may be employed in a method for assessing a risk of developing a disease state, detecting a disease state, and/or diagnosing a disease state. The method may include a somatic mutation in at least one gene in a set of genes. The genes may be obtained from a cell-free nucleic acid sample. The method then determines the disease state based on the detected somatic mutation. In various embodiments, detecting the somatic mutation can comprise detecting SNV, insertions, and/or deletions. In an embodiment, the method may also comprise developing a therapy, prognosis, or diagnosis in accordance with the gene and the somatic mutation detected at the gene.

In an embodiment, the set of genes may include three, five, or ten or more genes selected from a first group of genes. The first group of genes can comprise KRAS, TP53, ERBB2, EPHB1, NRAS, ACVR1B, TP63, KEAP1, CDK12, KMT2D, DICER1, TET2, LATS2, ETV5, GRIN2A, EPHA7, ASXL2, RET, CHD2, RB1, CDH1, PDGFRA, BRCA2, TFRC, ALK, KDM5A, SMAD4, ATR, NOTCH1, NRG1, CTNNB1, KMT2C, SNCAIP, MTOR, PIK3CA, SF3B1, NBN, LRP1B, TNFRSF14, ARID1A, INPP4A, ETS1, KAT6A, FBXW7, MGA, MYD88, CBL, BRAF, CREBBP, and APC.

In an embodiment, the set of genes can comprise. KRAS, TP53, ERBB2, EPHB1, NRAS, ACVR1B, TP63, and KEAP1. The set of genes may further comprise one or more genes selected from CDK12, KMT2D, DICER1, TET2, LAT52, ETV5, GRIN2A, EPHA7, ASXL2, and RET. The set of genes may further comprise one or more genes selected from TP53, NRAS, KMT2D, TET2, KMT2C, SF3B1, and LRP1B. The set of genes may further comprise one or more genes selected from MYD88, CBL, BRAF, CREBBP, and APC.

In an embodiment, the set of genes further comprises one or more genes from a second group of genes. The second group of genes are associated with hotspots for SNVs and indels. The second group of genes can include any of AKT1, ERBB3, IDH1, PTEN, ARAF, EZH2, IDH2, PTPRD, CD79A, FGFR3, MAP3K1, RHOA, CDKN2A, GATA3, MAPK1, RNF43, DNMT3A, GNAS, MSH2, SPTA1, EP300, HRAS, PREX2 and TERT.

In an embodiment, the set of genes further comprises one or more genes from a third group of genes. The third group of genes is associated with viral hotspots. The third group of genes can include any of HPV16, HPV18, EBV, and HBV.

In an embodiment, the method may be implemented by a non-transitory computer-readable medium. The medium can store one or more programs including instructions which, when executed by an electronic device including a processor, cause the device to perform any the method.

In an embodiment, an electronic device can comprise one or more processors, a memory and one or more programs for executing the method. That is, the electronic device comprises one or more programs stored in the memory and configured to be executed by the one or more processors. The programs include instructions for performing the method.

In an embodiment, any of the systems described herein may generate a cancer assay panel generated via the method. For example, a cancer assay panel can comprise one or more genes selected from a first group of genes associated with high signal cancers or liquid cancers, one or more genes selected from a second group of genes associated with hotspots for single nucleotide variants (SNVs) or indels, and one or more genes selected from a third group of genes associated with viral hotspots.

In an embodiment, first group of genes consists of: KRAS, TP53, ERBB2, EPHB1, NRAS, ACVR1B, TP63, KEAP1, CDK12, KMT2D, DICER1, TET2, LATS2, ETV5, GRIN2A, EPHA7, ASXL2, RET, CHD2, RB1, CDH1, PDGFRA, BRCA2, TFRC, ALK, KDM5A, SMAD4, ATR, NOTCH1, NRG1, CTNNB1, KMT2C, SNCAIP, MTOR, PIK3CA, SF3B1, NBN, LRP1B, TNFRSF14, ARID1A, INPP4A, ETS1, KAT6A, FBXW7, MGA, MYD88, CBL, BRAF, CREBBP, and APC.

In an embodiment, the second group of genes comprises a set of genes associated with hotspots for SNVs. The set of genes consists of AKT1, CDKN2A, DNMT3A, EP300, ERBB3, FGFR3, GNAS, HRAS, IDH1, IDH2, MAP3K1, MAPK1, PREX2, PTEN, PTPRD, RHOA, SPTA1, TERT, and EZH2. In an embodiment, the second group of genes comprises a set of genes associated with indels. The set of genes consists of ARAF, CD79A, GATA3, MSH2, PTEN, and RNF43. In an embodiment, the third group of genes consists of: HPV16, HPV18, EBV, and HBV.

In an embodiment, any of the systems, devices, or memories described herein may implement a method for generating a minimized cancer detection panel for determining a presence or absence of cancer in a patient. For example, a method can represent a workflow for generating the panel.

First, a system receives a request to generate a detection panel and including an aggregate kilobase size for the detection panel. The system then receives a plurality of genomic regions, with each genomic region associated with a likelihood that a variation in a feature of the genomic region is indicative of cancer. Each of the genomic regions has a kilobase size.

The system applies a classifier model to the plurality of genomic regions to generate the detection panel. The system employs the classifier model to determine a sensitivity score for each one of the genomic regions. The sensitivity score quantifies a contribution to a detection sensitivity of the detection panel. The detection sensitivity quantifies the likelihood that variations of the features in the set of genomic regions included in the cancer detection panel are indicative of cancer. In an embodiment, the variation of the feature that is indicative of cancer is a maximum variant allele frequency for the single nucleotide variant of the genomic region.

Next, the system employs the classifier model to rank the plurality of genomic regions according to their sensitivity score. Then the model selects, based on their rank, one or more of the genomic regions as the set of genomic regions for the detection panel. The sum of the kilobase sizes for set of genomic regions in the detection panel less than the aggregate kilobase size. In an embodiment, the determined set of genomic regions may be sent to the client device that transmitted the request. The set of genomic regions can be used to generate a panel employed to determine the presence of cancer in a patient.

In an embodiment, one or more of the genomic regions indicates a virus associated with cancer. The virus can be any of HPV16, HPV18, EBV, and HBV. In an embodiment, one or more of the genomic regions are associated with solid cancers. The genomic regions associated with solid cancers can be one of those disclosed in Table 1 and Table 2 herein. In an embodiment, one or more of the genomic regions are associated with liquid cancers. The genomic regions associated with liquid cancers can be one of those disclosed in Table 1 and Table 2 herein. In an embodiment, one or more of the genomic regions indicates a cancer hotspot. The genomic regions associated with cancer hotspots can be one of those disclosed in Table 3, Table 4, or Table 5 herein. In an embodiment, one or more of the genomic regions are associated with a specific type of cancer.

Because the set of genomic regions has less than a threshold kilobase size, in an embodiment, the detection panel includes fewer than 65, 55, or 45 genomic regions. Similarly, the aggregate kilobase size can be any of 390,000, 330,000, 270,000, 210,000, 150,000, or fewer kilobases.

In an embodiment, the request includes a type of cancer that the detection panel is designed to detect. In this case, the sensitivity score quantifies a contribution to a detection sensitivity of the detection panel for the type of cancer. Further, ranking the indicators further comprises ranking the genomic regions based on a type of cancer that the detection panel is designed to detect.

In an embodiment, one or more of the panels described herein comprises a set of probes designed to facilitate high quality detection assays. For example, a cancer assay panel can comprise at least a probe number of probe pairs. Each pair of the probe number of pairs comprises two probes configured to overlap each other by an overlapping sequence.

An overlapping sequence comprises an overlapping number of nucleobases. The overlapping sequence may be from a genomic indicator selected for the panel. Within the overlapping sequences, the overlapping number of nucleobases hybridizes a library molecule corresponding to one or more genomic regions. Each of the genomic regions has, for example, a maximum variant allele frequency for a single nucleotide variant of the genomic region. At least some of the variant allele frequencies for the genomic regions occurring in cancerous samples. Other somatic variations and quantifications of those variations are also possible.

In an embodiment, the cancerous samples are from subjects having cancer of a specific tissue of origin (“TOO”). The cancer of the specific TOO can be breast cancer, uterine cancer, cervical cancer, ovarian cancer, bladder cancer, renal urothelial cancer, renal cancer other than urothelial, prostate cancer, anorectal cancer, colorectal cancer, hepatobiliary cancer, pancreatic cancer, squamous upper gastrointestinal cancer, upper gastrointestinal cancer other than squamous, head and neck cancer, lung adenocarcinoma, small cell lung cancer, lung cancer other than adenocarcinoma or small cell lung cancer, neuroendocrine cancer, lung neuroendocrine tumors and other high-grade neuroendocrine tumors, melanoma, thyroid cancer, sarcoma, multiple myeloma, lymphoma, and leukemia.

In an embodiment, each of the probes comprises 70-140 nucleotides. Other numbers of nucleotides are also possible. In an embodiment, the probe number of probe pairs is 1000, 1500, 2000, 2500, or 3000 probe pairs. In an embodiment, the overlapping number of nucleobases in the overlapping sequence is 20, 30, 40, 50, 60, 70, or 80 nucleobases.

In an embodiment, the cancer assay panel includes least 2900 probes selected by a classifier model as disclosed herein. The classifier model selects the at least 2900 probes based on a sensitivity score quantifying a detection sensitivity for each of the 2900 probes. The at least 2900 probes have an aggregate kilobase size less than a target kilobase size. In this case, the classifier model selects the 2900 probes with the highest sensitivity scores while remaining below the target kilobase size.

In an embodiment, one or more of the genomic regions is in Table 1, Table 2, Table 3, Table 4, or Table 5 disclosed herein. In an embodiment, one or more of the genomic regions are associated with a viral region, a viral region indicating a virus sequence associated with cancer.

### BRIEF DESCRIPTION OF DRAWINGS

FIG. 1 is flowchart of a method for preparing a nucleic acid sample for sequencing according to one embodiment.

FIG. 2A is block diagram of a processing system for processing sequence reads according to one embodiment.

FIG. 2B is a block diagram of a panel generator for generating panels according to one embodiment.

FIG. 3 is flowchart of a method for determining variants of sequence reads according to one embodiment.

FIG. 4 is a flow chart of a workflow for generating a disease detection panel according to one embodiment.

FIG. 5 illustrates a receiver operating characteristic plot showing performance of three classifiers based on a panel that includes a large set of genomic regions (approximately 2 Mb) not identified or selected in the manners described herein.

FIG. 6A illustrates a ROC plot for panels generated by a bi-classifier and mono-classifier that are applied to training data according to embodiment.

FIG. 6B illustrates a ROC result plot for the ROC plot in FIG. 6A according to one embodiment.

FIG. 6C illustrates a ROC plot for panels generated by a bi-classifier and mono classifier that are applied to real data according to one embodiment.

FIG. 6D illustrates a ROC result plot for the ROC plot of FIG. 6C according to one embodiment.

FIG. 7A illustrates a ROC plot for panels generated by a bi-classifier and mono-classifier that are applied to training samples according to one embodiment.

FIG. 7B illustrates a ROC result plot for the ROC plot of FIG. 7A according to one embodiment.

FIG. 7C illustrates a ROC plot for panels generated by a bi-classifier and mono classifier that are applied to test samples according to one embodiment.

FIG. 7D illustrates a ROC results plot of the ROC plot in FIG. 7C according to one embodiment.

FIG. 8A illustrates a coefficient plot for solid cancers according to one embodiment.

FIG. 8B illustrates a cancerous frequency plot for solid cancers according to one embodiment.

FIG. 8C illustrates a non-cancerous frequency plot for solid cancers according to one embodiment.

FIG. 9A illustrates a coefficient plot for liquid cancers according to one embodiment.

FIG. 9B illustrates a cancerous frequency plot for liquid cancers according to one embodiment.

FIG. 9C illustrates a non-cancerous frequency plot for liquid cancers according to one embodiment.

FIG. 10 illustrates a coefficient plot for solid and liquid cancers according to one embodiment.

FIG. 11A shows a detection contribution plot for solid cancers according to one embodiment.

FIG. 11B shows a detection contribution plot for liquid cancers according to one embodiment.

FIG. 12 shows a size contribution plot for solid cancers according to one embodiment.

FIG. 13A shows a coverage plot according to one embodiment.

FIG. 13B shows a coverage size plot according to one embodiment.

FIG. 14 shows a type classification plot according to one embodiment.

FIG. 15 shows an accuracy contribution plot for a panel according to one embodiment.

FIG. 16 shows an example workflow for generating a panel for determining a cancer presence according to one embodiment.

FIG. 17A is a population plot for a set of training data according to one embodiment.

FIG. 17B is a sensitivity plot according to one example embodiment.

FIG. 18A is a population plot for a set of test data according to one embodiment.

FIG. 18B is a sensitivity plot according to one example embodiment.

FIG. 19 shows an example workflow for generating a panel less than a threshold panel seize according to one embodiment.

FIG. 20A shows an SNV count plot for different cancer types for a large set panel according to one embodiment.

FIG. 20B shows an SNV count plot for different cancer stages for a large set panel according to one embodiment.

FIG. 20C shows an SNV count plot for different cancer types for a panel generated using the panel generator according to one embodiment.

FIG. 20D shows an SNV count plot for different cancer stages for a panel generated using the panel generator according to one embodiment.

FIG. 20E shows an SNV difference plot for different cancer types for a large set panel according to one embodiment.

FIG. 20F shows an SNV difference plot for different cancer stages for a panel generated using the panel generator according to one embodiment.

FIG. 21A shows an indel count plot for different cancer types for a large set panel according to one embodiment.

FIG. 21B shows an indel count plot for different cancer stages for a large set panel according to one embodiment.

FIG. 21C shows an indel count plot for different cancer types for a panel generated using the panel generator according to one embodiment.

FIG. 21D shows an indel count plot for different cancer stages for a panel generated using the panel generator according to one embodiment.

FIG. 21E shows an indel difference plot for different cancer types for a large set panel according to one embodiment.

FIG. 21F shows an indel difference plot for different cancer stages for a panel generated using the panel generator according to one embodiment.

### DETAILED DESCRIPTION

### I. Definitions

The term “individual” refers to a human individual. The term “healthy individual” refers to an individual presumed to not have a cancer or disease. The term “subject” refers to an individual who is known to have, or potentially has, a cancer or disease.

The term “sequence reads” refers to nucleobase sequences read from a sample obtained from an individual. Sequence reads can be obtained through various methods known in the art.

The term “read segment” or “read” refers to any nucleobase sequences including sequence reads obtained from an individual and/or nucleobase sequences derived from the initial sequence read from a sample obtained from an individual. For example, a read segment can refer to an aligned sequence read, a collapsed sequence read, or a stitched read. Furthermore, a read segment can refer to an individual nucleobase base, such as a single nucleobase variant.

The term “single nucleobase variant” or “SNV” refers to a substitution of one nucleobase to a different nucleobase at a position (e.g., site) of a nucleobase sequence, e.g., a sequence read from an individual. A substitution from a first nucleobase X to a second nucleobase Y can be denoted as “X>Y.” For example, a cytosine to thymine SNV can be denoted as “C>T.”

The term “indel” refers to any insertion or deletion of one or more base pairs having a length and a position (which can also be referred to as an anchor position) in a sequence read. An insertion corresponds to a positive length, while a deletion corresponds to a negative length.

The term “mutation” refers to one or more SNVs or indels.

The term “true positive” refers to a mutation that indicates real biology, for example, presence of a potential cancer, disease, or germline mutation in an individual. True positives are not caused by mutations naturally occurring in healthy individuals (e.g., recurrent mutations) or other sources of artifacts such as process errors during assay preparation of nucleic acid samples.

The term “false positive” refers to a mutation incorrectly determined to be a true positive. Generally, false positives can be more likely to occur when processing sequence reads associated with greater mean noise rates or greater uncertainty in noise rates.

The term “cell-free nucleic acid,” “cell-free DNA,” or “cfDNA” refers to nucleic acid fragments that circulate in an individual's body (e.g., bloodstream) and originate from one or more healthy cells and/or from one or more cancer cells. cfDNA can be obtained from a blood sample.

The term “circulating tumor DNA” or “ctDNA” refers to nucleic acid fragments that originate from tumor cells or other types of cancer cells, which can be released into an individual's bloodstream as result of biological processes such as apoptosis or necrosis of dying cells or actively released by viable tumor cells. In some cases, ctDNA is DNA found in cfDNA.

The term “genomic nucleic acid,” “genomic DNA,” or “gDNA” refers to nucleic acid including chromosomal DNA that originates from one or more healthy cells. In some cases, white blood cells are assumed to be healthy cells.

The term “white blood cell DNA,” or “wbcDNA” refers to nucleic acid including chromosomal DNA that originates from white blood cells. Generally, wbcDNA is gDNA and is assumed to be healthy DNA.

The term “tissue nucleic acid,” “cancerous tissue DNA,” or “tDNA” refers to nucleic acid including chromosomal DNA from tumor cells or other types of cancer cells that are obtained from cancerous tissue or a tumor. In some cases, tDNA is obtained from a biopsy of a tumor.

The term “alternative allele” or “ALT” refers to an allele having one or more mutations relative to a reference allele, e.g., corresponding to a known gene.

The term “sequencing depth” or “depth” refers to a total number of read segments from a sample obtained from an individual.

The term “alternate depth” or “AD” refers to a number of read segments in a sample that support an ALT, e.g., include mutations of the ALT.

The term “alternate frequency” or “AF” refers to the frequency of a given ALT. The AF can be determined by dividing the corresponding AD of a sample by the depth of the sample for the given ALT.

### II. Example Assay Protocol

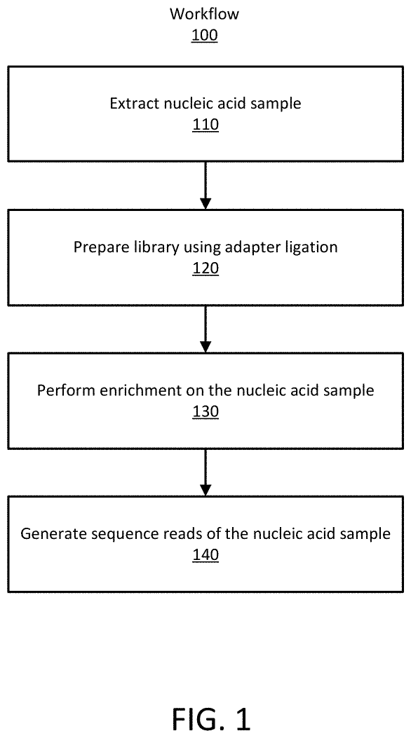

FIG. 1 is flowchart of a method for preparing a nucleic acid sample for sequencing according to one embodiment. The workflow 100 includes, but is not limited to, the following steps. For example, any step of the workflow 100 can comprise a quantitation sub-step for quality control or other laboratory assay procedures known to one skilled in the art.

In step 110, a nucleic acid sample (DNA or RNA) is extracted from a subject. In the present disclosure, DNA and RNA can be used interchangeably unless otherwise indicated. That is, the following embodiments for using error source information in variant calling and quality control can be applicable to both DNA and RNA types of nucleic acid sequences. However, the examples described herein can focus on DNA for purposes of clarity and explanation. The sample can be any subset of the human genome, including the whole genome. The sample can be extracted from a subject known to have or suspected of having cancer. The sample can include blood, plasma, serum, urine, fecal, saliva, other types of bodily fluids, or any combination thereof. In some cases, the sample can include tissue or bodily fluids extracted from tissue. In some embodiments, methods for drawing a blood sample (e.g., syringe or finger prick) can be less invasive than procedures for obtaining a tissue biopsy, which can require surgery. The extracted sample can include cfDNA and/or ctDNA. For healthy individuals, the human body can naturally clear out cfDNA and other cellular debris. If a subject has a cancer or disease, ctDNA in an extracted sample can be present at a detectable level for diagnosis.

Additionally, the extracted sample can include wbcDNA. Extracting the nucleic acid sample can further include separating the cfDNA and/or ctDNA from the wbcDNA. Extracting the wbcDNA from the cfDNA and/or ctDNA can occur when the DNA is separated from the sample. In the case of a blood sample, the wbcDNA is obtained from a buff coat fraction of the blood sample. The wbcDNA can be sheared to obtain wbcDNA fragments less than 300 base pairs in length. Separating the wbcDNA from the cfDNA and/or ctDNA allows the wbcDNA to be sequenced independently from the cfDNA and/or ctDNA. Generally the sequencing process for wbcDNA is similar to the sequencing process for cfDNA and/or ctDNA.

In step 120, a sequencing library is prepared. During library preparation, unique molecular identifiers (UMI) are added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment, which provides a way to identify sequence reads that came from the same original fragment in downstream analysis.

In step 130, targeted DNA sequences are enriched from the library. During enrichment, hybridization probes (also referred to herein as “probes”) are used to target, and pull down, nucleic acid fragments informative for the presence or absence of cancer (or disease), cancer status, or a cancer classification (e.g., cancer type or tissue of origin). For a given workflow, the probes can be designed to anneal (or hybridize) to a target (complementary) strand of DNA or RNA. The target strand can be the “positive” strand (e.g., the strand transcribed into mRNA, and subsequently translated into a protein) or the complementary “negative” strand. The probes can range in length from 10s, 100s, or 1000s of base pairs. In one embodiment, the probes are designed based on a gene panel to analyze particular mutations or target regions of the genome (e.g., of the human or another organism) that are suspected to correspond to certain cancers or other types of diseases. Moreover, the probes can cover overlapping portions of a target region. By using a targeted gene panel rather than sequencing all expressed genes of a genome, also known as “whole exome sequencing,” the workflow 100 can be used to increase sequencing depth of the target regions, where depth refers to the count of the number of times a given target sequence within the sample has been sequenced. Increasing sequencing depth reduces required input amounts of the nucleic acid sample. After a hybridization step, the hybridized nucleic acid fragments are captured and can also be amplified using PCR.

In step 140, sequence reads are generated from the enriched DNA sequences. Sequencing data can be acquired from the enriched DNA sequences by known means in the art. For example, the workflow 100 can include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators. In other embodiments, sequences can be detected using amplification based detection or methylation-specific amplification means, such as, detection by polymerase chain reaction (PCR), digital PCR (dPCR), quantitative PCR (qPCR), real time PCR (RT-PCR), quantitative real time PCR (qRT-PCR), or other well-known means in the art.

In some embodiments, the sequence reads can be aligned to a reference genome using known methods in the art to determine alignment position information. The alignment position information can indicate a beginning position and an end position of a region in the reference genome that corresponds to a beginning nucleobase base and end nucleobase base of a given sequence read. Alignment position information can also include sequence read length, which can be determined from the beginning position and end position. A region in the reference genome can be associated with a gene or a segment of a gene. As cfDNA and/or ctDNA and wbcDNA are sequenced independently, sequence reads for both cfDNA and or ctDNA and wbcDNA are independently generated.

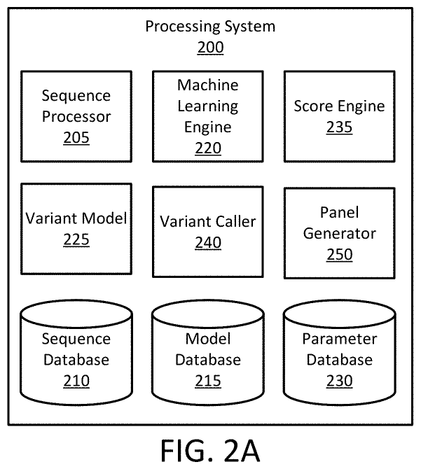

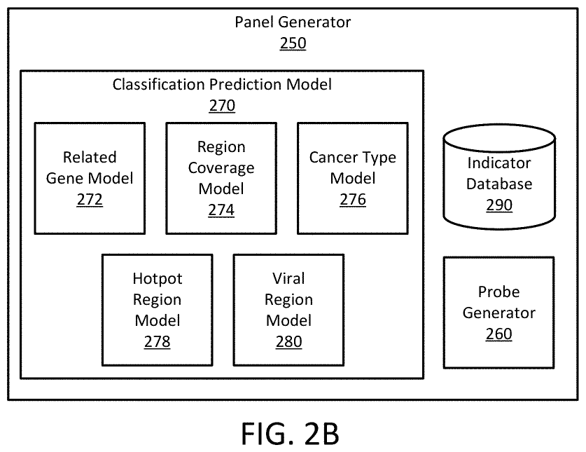

In various embodiments, a sequence read is comprised of a read pair denoted as R₁ and R₂. For example, the first read R₁ can be sequenced from a first end of a nucleic acid fragment whereas the second read R₂ can be sequenced from the second end of the nucleic acid fragment. Therefore, nucleobase base pairs of the first read R₁ and second read R₂ can be aligned consistently (e.g., in opposite orientations) with nucleobase bases of the reference genome. Alignment position information derived from the read pair R₁ and R₂ can include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary) format can be generated and output for further analysis such as variant calling, as described below with respect to FIG. 2.

### III. Example Processing System

FIG. 2A is block diagram of a processing system 200 for processing sequence reads and generating disease detection panels according to one embodiment. The processing system 200 includes a sequence processor 205, sequence database 210, model database 215, machine learning engine 220, models 225 (for example, including one or more Bayesian hierarchical models or joint models), parameter database 230, score engine 235, variant caller 240, and a panel generator 250. FIG. 2B illustrates a block diagram of a panel generator for generating panels according to one embodiment. The panel generator 250 includes a classification prediction model 270, an indicator database 290, and a probe generator 260.

III.A Determining Variants from Sequences

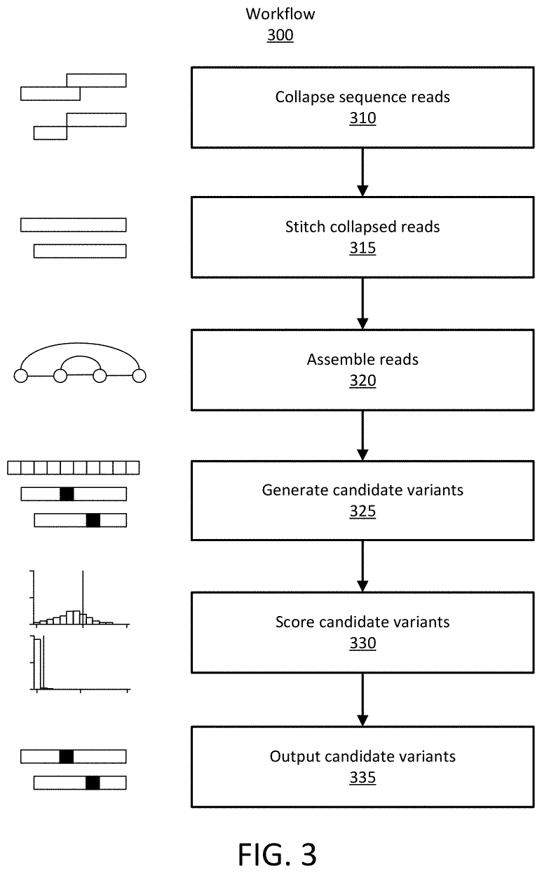

FIG. 3 is a flowchart of a workflow for determining variants of sequence reads according to one embodiment. In some embodiments, the processing system 200 performs the workflow 300 to perform variant calling (e.g., for SNVs and/or indels) based on input sequencing data. Further, the processing system 200 can obtain the input sequencing data from an output file associated with nucleic acid sample prepared using the workflow 100 described above. The workflow 300 includes, but is not limited to, the following steps, which are described with respect to the components of the processing system 200. In other embodiments, one or more steps of the workflow 300 can be replaced by a step of a different process for generating variant calls, e.g., using Variant Call Format (VCF), such as HaplotypeCaller, VarScan, Strelka, or SomaticSniper.

At step 310, the sequence processor 205 collapses aligned sequence reads of the input sequencing data. In one embodiment, collapsing sequence reads includes using UMIs, and optionally alignment position information from sequencing data of an output file (e.g., from the workflow 100 shown in FIG. 1) to collapse multiple sequence reads into a consensus sequence for determining the most likely sequence of a nucleic acid fragment or a portion thereof. Since the UMIs are replicated with the ligated nucleic acid fragments through enrichment and PCR, the sequence processor 205 can determine that certain sequence reads originated from the same molecule in a nucleic acid sample. In some embodiments, sequence reads that have the same or similar alignment position information (e.g., beginning and end positions within a threshold offset) and include a common UMI are collapsed, and the sequence processor 205 generates a collapsed read (also referred to herein as a consensus read) to represent the nucleic acid fragment. The sequence processor 205 designates a consensus read as “duplex” if the corresponding pair of collapsed reads have a common UMI, which indicates that both positive and negative strands of the originating nucleic acid molecule is captured; otherwise, the collapsed read is designated “non-duplex.” In some embodiments, the sequence processor 205 can perform other types of error correction on sequence reads as an alternate to, or in addition to, collapsing sequence reads.

At step 315, the sequence processor 205 stitches the collapsed reads based on the corresponding alignment position information. In some embodiments, the sequence processor 205 compares alignment position information between a first read and a second read to determine whether nucleobase base pairs of the first and second reads overlap in the reference genome. In one use case, responsive to determining that an overlap (e.g., of a given number of nucleobase bases) between the first and second reads is greater than a threshold length (e.g., threshold number of nucleobase bases), the sequence processor 205 designates the first and second reads as “stitched”; otherwise, the collapsed reads are designated “unstitched.” In some embodiments, a first and second read are stitched if the overlap is greater than the threshold length and if the overlap is not a sliding overlap. For example, a sliding overlap can include a homopolymer run (e.g., a single repeating nucleobase base), a dinucleobase run (e.g., two-nucleobase base sequence), or a trinucleobase run (e.g., three-nucleobase base sequence), where the homopolymer run, dinucleobase run, or trinucleobase run has at least a threshold length of base pairs.

At step 320, the sequence processor 205 assembles reads into paths. In some embodiments, the sequence processor 205 assembles reads to generate a directed graph, for example, a de Bruijn graph, for a target region (e.g., a gene). Unidirectional edges of the directed graph represent sequences of k nucleobase bases (also referred to herein as “k-mers”) in the target region, and the edges are connected by vertices (or nodes). The sequence processor 205 aligns collapsed reads to a directed graph such that any of the collapsed reads can be represented in order by a subset of the edges and corresponding vertices.

In some embodiments, the sequence processor 205 determines sets of parameters describing directed graphs and processes directed graphs. Additionally, the set of parameters can include a count of successfully aligned k-mers from collapsed reads to a k-mer represented by a node or edge in the directed graph. The sequence processor 205 stores, e.g., in the sequence database 210, directed graphs and corresponding sets of parameters, which can be retrieved to update graphs or generate new graphs. For instance, the sequence processor 205 can generate a compressed version of a directed graph (e.g., or modify an existing graph) based on the set of parameters. In one use case, in order to filter out data of a directed graph having lower levels of importance, the sequence processor 205 removes (e.g., “trims” or “prunes”) nodes or edges having a count less than a threshold value, and maintains nodes or edges having counts greater than or equal to the threshold value.

At step 325, the variant caller 240 generates candidate variants from the paths assembled by the sequence processor 205. In one embodiment, the variant caller 240 generates the candidate variants by comparing a directed graph (which can have been compressed by pruning edges or nodes in step 310) to a reference sequence of a target region of a genome. The variant caller 240 can align edges of the directed graph to the reference sequence, and records the genomic positions of mismatched edges and mismatched nucleobase bases adjacent to the edges as the locations of candidate variants. Additionally, the variant caller 240 can generate candidate variants based on the sequencing depth of a target region. In particular, the variant caller 240 can be more confident in identifying variants in target regions that have greater sequencing depth, for example, because a greater number of sequence reads help to resolve (e.g., using redundancies) mismatches or other base pair variations between sequences.

In one embodiment, the variant caller 240 generate candidate variants using a variant model 225 to determine expected noise rates for sequence reads from a subject. The variant model 225 can be a Bayesian hierarchical model, though in some embodiments, the processing system 200 uses one or more different types of models. Moreover, a Bayesian hierarchical model can be one of many possible model architectures that can be used to generate candidate variants and which are related to each other in that they all model position-specific noise information in order to improve the sensitivity/specificity of variant calling. More specifically, the machine learning engine 220 trains the variant model 225 using samples from healthy individuals to model the expected noise rates per position of sequence reads.

Further, multiple different models can be stored in the model database 215 or retrieved for application post-training. For example, a first model is trained to model SNV noise rates and a second model is trained to model indel noise rates. Further, the score engine 235 can use parameters of the variant model 225 to determine a likelihood of one or more true positives in a sequence read. The score engine 235 can determine a quality score (e.g., on a logarithmic scale) based on the likelihood. For example, the quality score is a Phred quality score Q=−10·log₁₀ P, where P is the likelihood of an incorrect candidate variant call (e.g., a false positive).

At step 330, the score engine 235 scores the candidate variants based on the variant model 225 or corresponding likelihoods of true positives or quality scores.

At step 335, the processing system 200 outputs the candidate variants. In some embodiments, the processing system 200 outputs some or all of the determined candidate variants along with the corresponding scores. Downstream systems, e.g., external to the processing system 200 or other components of the processing system 200, can use the candidate variants and scores for various applications including, but not limited to, predicting presence of cancer, disease, or germline mutations.

Candidate variants are outputted for both cfDNA and/or ctDNA and wbcDNA. Herein, generally, candidate variants for wbcDNA are “normals” while candidate variants for cfDNA and/or ctDNA are “variants.” Various detection methods and models can compare variants to normals to determine if the variants include signatures of cancer or any other disease. In various embodiments, normals and variants can be generated using any other process, any number of samples (e.g., a tumor biopsy or blood sample), or accessed from a database storing candidate variants.

### III.B Generating a Panel

Returning to FIG. 2B, the panel generator 250 generates a disease detection panel using various features, scores, sequences, etc. determined by the processing system 200. One example disease detection panel described herein is a cancer detection panel, but the disease detection panel can also detect other diseases.

The panel generator 250 includes an indicator database 290 that stores genomic regions. More specifically, the indicator database 290 stores sequencing data (e.g., variants and normals) which can be used to detect presence or absence of cancer signal(s) in a sample from a subject, and/or otherwise predict a likelihood that a subject has cancer. Sequencing data can be associated and stored with its corresponding genomic region. The indicator database can also store sequencing data processed by the system 200, but can also store sequencing data not processed by the system 200, such as sequencing data uploaded from an external source and/or otherwise retrieved from external or publicly available databases. Genomic regions stored in the indicator database 290 are described in more detail below.

The panel generator 250 employs a classification prediction model 270 (“classification model”) to identify genomic regions to include in a panel. The classification model 270 predicts the classification capability of a panel including identified genomic regions. The process of identifying and selecting genomic regions for a panel is described in more detail below.

The classification model 270 can employ different models that identify different types of genomic regions. To illustrate, the classification model 270 can identify (i) genomic regions of cancer related genes using a related gene model 272, (ii) indicative genomic regions in cancerous samples using a region coverage model 274, (iii) genomic regions indicating cancer type using a cancer type model 276, (iv) hotspot genomic regions using a hotspot region model 278, and (v) viral genomic regions associated with cancer using a viral region model 280. The various models are described below.

The panel generator 250 also includes a probe generator 260. The probe generator 260 determines cancer detection probes for genomic regions identified for a panel. The probe generator 260 is described in more detail below.

### IV. Variants that are Indicative of Cancer

The indicator database 290 includes sets of genomic regions that can be indicative of a disease presence (“indicator set”). Each indicator set can include sequences obtained from different sample types, via different processes, etc. For example, a first indicator set can include sequences obtained from both cancerous samples and non-cancerous samples, while a second indicator set can include sequences obtained from only cancerous samples. In another example, a first indicator set can include both sequences obtained from solid cancers and liquid cancers, while a second indicator set can include sequences obtained from only solid cancers. It is noted that a detection panel generated by the panel generator 250 can include one or more indicator sets, in any combination and in part or in whole, as described below.

Some indicator sets are selected from established indicator libraries. For example, an indicator set can include one or more genomic regions selected from an indicator library of genes identified in The Circulating Cell-free Genome Atlas Study (“CCGA”; Clinical Trial.gov identifier NCT02889978). The CCGA Study is a prospective, observational, longitudinal, study designed to characterize the landscape of genomic cancer signals in the blood of people with and without cancer. De-identified biospecimens were collected from approximately 15,000 participants from 142 sites across the United States and Canada. Samples were selected to ensure a prespecified distribution of cancer types and non-cancers across sites in each cohort, and cancer and non-cancer samples were frequency age-matched by gender. Table 1 lists an example CCGA indicator set comprising 50 genomic regions or genes selected from the CCGA Study, in accordance with various embodiments described herein.

TABLE 1
50 CCGA genomic regions.

| | | | | | |
|---|---|---|---|---|---|
| KRAS | KMT2D | CHD2 | ATR | NBN | MYD88 |
| TP53 | DICER1 | RB1 | NOTCH1 | LRP1B | CBL |
| ERBB2 | TET2 | CDH1 | NRG1 | TFRSF14 | BRAF |
| EPHB1 | LATS2 | PDGFRA | CTNNB1 | ARID1A | CREBBP |
| NRAS | ETV5 | BRCA2 | KMT2C | INPP4A | APC |
| ACVR1B | GRIN2A | TFRC | SNCAIP | ETS1 | SMAD4 |
| TP63 | EPHA7 | ALK | MTOR | KAT6A | SF3B1 |
| KEAP1 | ASXL2 | KDM5A | PIK3CA | FBXW7 | MGA |
| CDK12 | RET | | | | |

In another example, an indicator set can include one or more genomic regions selected from a publicly available database, such as the database of genes identified in The Cancer Genome Atlas Program (“TCGA”; Clinical Trial.gov identifier NCT02889978). The TCGA database is a public resource developed through a collaboration between the National Cancer Institute (NCI) and the National Human Genome Research Institute (NHGRI) that molecularly characterized over 20,000 primary cancer and matched normal samples spanning 33 cancer types. Table 2 lists an example TCGA indicator set comprising 19 genomic regions or genes selected from TCGA, in accordance with various embodiments described herein.

TABLE 2
19 TCGA genomic regions.

| | | | | | |
|---|---|---|---|---|---|
| CDH10 | CSMD3 | DCDC1 | FAM135B | ZNF536 | BRINP3 |
| NFE2L2 | HCN1 | SPTA1 | CNTNAP5 | PCDH11X | CDH9 |
| RYR2 | PAPPA2 | NPAP1 | DCAF4L2 | ZNF479 | PCDH10 |
| COL11A1 | | | | | |

In another example, an indicator set can include genomic regions with particular sequences (“mutation hotspots”) indicative of cancer. In some examples, such hotspots sites can be found in literature, publicly available platforms of cancer data such as the Genomic Data Commons Data Portal (“GDC”), and/or corroborated with other studies such as the CCGA Study described above. For instance, a promoter hotspot site in EZH2 that was frequently mutated across CCGA patients can be included or otherwise considered for inclusion in a detection panel. Table 3 lists an example hotspot indicator set comprising 18 genomic regions with hotspots indicative of cancer. The number in the parenthesis indicates the number of hotspot sites in that gene or genomic region indicative of cancer.

TABLE 3
18 hotspot genomic regions with hotspot sites.

AKT (1)
CDKN2A (6)
DNMT3A (2)
EP300 (1)
ERBB3 (1)
FGFR3 (1)
GNAS (1)
HRAS (1)
IDH1 (2)
IDH2 (1)
MAP3K1 (1)
MAPK1 (1)
PREX2 (1)
PTEN (2)
PTRD (1)
RHOA (1)
SPTA (1)
EZH2 (1)

In another example, an indicator set can include genomic regions comprising SNVs and/or indels whose mutation is indicative of cancer (“List A”). Table 4 lists 24 genomic regions for the List A indicator set. The letter in parenthesis indicates whether the genomic region comprises one or more SNVs (S), one or more indels (I), or both. One or more of the genomic regions in the List A indicator set can be included in a detection panel in accordance with various embodiments. In some examples, only the genomic regions corresponding to SNVs are included in the detection panel.

TABLE 4
List A Genomic Regions

AKT1 (S)
ARAF (I)
CD79A (I)
CDKN2A (S)
DNMT3A (S)
EP300 (S)
ERBB3 (S)
EZH2 (S)
FGFR3 (S)
GATA3 (I)
GNAS (S)
HRAS (S)
IDH1 (S)
IDH2 (S)
MAP3K1 (S)
MAPK1 (S)
MSH2 (I)
PREX2 (S)
PTEN (I) (S)
PTPRD (S)
RHOA (S)
RNF43 (I)
SPTA1 (S)
TERT (S)

In another example, another indicator set can include genomic regions comprising SNVs and/or indels whose mutation is indicative of cancer (“List B”). Table 5 lists 64 genomic regions for the List B indicator set. The letter in parenthesis indicates whether the genomic region comprises one or more SNVs (S), one or more indels (I), or both. One or more of the genomic regions in the List B indicator set can be included in a detection panel in accordance with various embodiments. In some examples, only the genomic regions corresponding to SNVs are included in the detection panel.

TABLE 5
List B Genomic Regions

AKT1 (S)
AMER1 (S) (I)
ARAF (I)
ARID2 (S)
ASXL1 (I)
BARD1 (I)
BCOR (S)
BCORL1 (I)
CARD11 (I)
CD79A (I)
CDKN2A (S)
CYLD (I)
DDR2 (S)
DNMT1 (S)
DNMT3A (S)
EP300 (S)
EPHA3 (I)
EPHA5 (S)
ERBB3 (S)
ERBB4 (S) (I)
EZH2 (S)
FGF14 (S)
FGFR1 (S)
FGFR3 (S)
FLT4 (I)
GATA3 (S) (I)
GLI1 (I)
GNAQ (S)
GNAS (S)
HRAS (S)
IDH1 (S)
IDH2 (S)
IL7R (I)
KDR (S)
KLHL6 (S)
KMT2B (I)
MAP2K1 (S)
MAP3K1 (S)
MAPK1 (S)
MSH2 (I)
MSH6 (S)
NF1 (S)
NSD1 (I)
NTRK1 (S)
PBRM1 (S) (I)
PIK3R3 (I)
POLE (S)
PREX2 (S)
PRKDC (S) (I)
PTEN (S) (I)
PTPRD (S)
PTPRT (S) (I)
RHOA (S)
RNF43 (I)
SLIT2 (S)
SOX9 (I)
SPTA1 (S)
STK11 (I)
TAF1 (S)
TCF7L2 (S)
TERT (S)
TET1 (I)
TOP2A (I)
ZFHX3 (I)

In another example, another indicator set can include genomic regions comprising SNVs and/or indels whose mutation is indicative of cancer (“List C”). Table 6 lists 153 genomic regions for the List C indicator set. The letter in parenthesis indicates whether the genomic region comprises one or more SNVs (S), one or more indels (I), or both. One or more of the genomic regions in the List C indicator set can be included in a detection panel in accordance with various embodiments. In some examples, only the genomic regions corresponding to SNVs are included in the detection panel.

TABLE 6
List C genomic regions

| | | | | |
|---|---|---|---|---|
| AKT1 (S) | EPHA3 (I) | INSRR (S) | NF1 (S) | RASA1 (S) (I) |
| AMER1 (S) (I) | EPHA5 (S) | IRF2 (S) | NPM1 (I) | RHOA (S) |
| ARAF (I) | ERBB3 (S) | IRF2 (I) | NSD1 (I) | RICTOR (S) |
| ARID2 (S) | ERBB4 (S) (I) | JAK1 (I) | NTRK1 (S) | RNF43 (I) |
| ARID5B (I) | ERCC2 (S) | KDM6A (S) | NUP93 (S) | RUNX1T1 (S) |
| ASXL1 (I) | ESR1 (S) | KDR (S) | PAK7 (S) | SLIT2 (S) |
| ATM (S) (I) | ETV1 (S) | KIF5B (I) | PALB2 (I) | SLX4 (I) |
| ATRX (S) (I) | EZH2 (S) | KIT (S) | PAX3 (S) | SMAD2 (S) |
| AXIN2 (I) | FAS (I) | KLHL6 (S) | PAX7 (S) | SMARCA4 (S) |
| B2M (S) (I) | FAT1 (S) | KMT2B (I) | PBRM1 (S) (I) | SMO (I) |
| BARD1 (I) | FGF14 (S) | LATS1 (S) | PGR (S) | SOX17 (S) |
| BCL6 (S) | FGFR1 (S) | LYN (I) | PIK3R1 (S) (I) | SOX9 (I) |
| BCOR (S) | FGFR2 (S) | LZTR1 (S) | PIK3R2 (S) | SPEN (I) |
| BCORL1 (I) | FGFR3 (S) | MAP2K1 (S) | PIK3R3 (I) | SPOP (S) |
| BLM (I) | FLT3 (S) | MAP3K1 (S) | PLK2 (I) | SPTA1 (S) |
| CARD11 (I) | FLT4 (I) | MAP3K4 (I) | PMS1 (I) | STAG2 (S) |
| CD79A (I) | FUBP1 (S) (I) | MAPK1 (S) | POLE (S) | STAT5B (I) |
| CDC73 (S) | FYN (S) | MAX (S) | PPARG (I) | STK11 (I) |
| CDKN2A (S) | GATA3 (S) (I) | MEN1 (I) | PPM1D (S) (I) | SYNE1 (S) |
| CHD4 (S) (I) | GLI1 (I) | MET (S) | PPP2R1A (S) | TAF1 (S) |
| CIC (S) (I) | GNA11 (S) | MLLT3 (I) | PPP6C (S) | TCF7L2 (S) |
| CSF3R (I) | GNAQ (S) | MRE11A(I) | PREX2 (S) | TERT (S) |
| CTCF (S) (I) | GNAS (S) | MSH2 (I) | PRKDC (S) (I) | TET1 (I) |
| CTNNA1 (S) | H3F3C (S) | MSH3 (I) | PTCH1 (I) | TGFBR2 (S) |
| CYLD (I) | HIST1H3B (S) | MSH6 (S) | PTEN (S) (I) | TOP2A (I) |
| DDR2 (S) | HIST1H3C (S) | MST1 (S) | PTPN11 (S) | TSC1 (I) |
| DIS3 (S) | HNF1A (I) | MYB (I) | PTPRD (S) | XPO1 (S) |
| DNMT1 (S) | HRAS (S) | MYC (S) | PTPRT (S) (I) | XRCC2 (I) |
| DNMT3A (S) | IDH1 (S) | MYCN (S) (I) | QKI (I) | ZFHX3 (I) |
| EML4 (I) | IDH2 (S) | NAB2 (I) | RAC1 (S) | RASA1 (S) (I) |
| EP300 (S) (I) | IL7R (I) | NCOR1 (I) | RAF1 (S) | RHOA (S) |
| AKT1 (S) | EPHA3 (I) | INSRR (S) | NF1 (S) | RICTOR (S) |
| AMER1 (S) (I) | EPHA5 (S) | IRF2 (S) | NPM1 (I) | RNF43 (I) |
| ARAF (I) | ERBB3 (S) | IRF2 (I) | NSD1 (I) | RUNX1T1 (S) |
| ARID2 (S) | ERBB4 (S) (I) | JAK1 (I) | NTRK1 (S) | SLIT2 (S) |
| ARID5B (I) | ERCC2 (S) | KDM6A (S) | NUP93 (S) | SLX4 (I) |
| ASXL1 (I) | ESR1 (S) | KDR (S) | PAK7 (S) | SMAD2 (S) |
| ATM (S) (I) | ETV1 (S) | KIF5B (I) | PALB2 (I) | SMARCA4 (S) |
| ATRX (S) (I) | EZH2 (S) | KIT (S) | PAX3 (S) | SMO (I) |
| AXIN2 (I) | FAS (I) | KLHL6 (S) | PAX7 (S) | SOX17 (S) |
| B2M (S) (I) | FAT1 (S) | KMT2B (I) | PBRM1 (S) (I) | SOX9 (I) |
| BARD1 (I) | FGF14 (S) | LATS1 (S) | PGR (S) | SPEN (I) |
| BCL6 (S) | FGFR1 (S) | LYN (I) | PIK3R1 (S) (I) | SPOP (S) |
| BCOR (S) | FGFR2 (S) | LZTR1 (S) | PIK3R2 (S) | SPTA1 (S) |
| BCORL1 (I) | FGFR3 (S) | MAP2K1 (S) | PIK3R3 (I) | STAG2 (S) |
| BLM (I) | FLT3 (S) | MAP3K1 (S) | PLK2 (I) | STAT5B (I) |
| CARD11 (I) | FLT4 (I) | MAP3K4 (I) | PMS1 (I) | STK11 (I) |
| CD79A (I) | FUBP1 (S) (I) | MAPK1 (S) | POLE (S) | SYNE1 (S) |
| CDC73 (S) | FYN (S) | MAX (S) | PPARG (I) | TAF1 (S) |
| CDKN2A (S) | GATA3 (S) (I) | MEN1 (I) | PPM1D (S) (I) | TCF7L2 (S) |
| CHD4 (S) (I) | GLI1 (I) | MET (S) | PPP2R1A (S) | TERT (S) |
| CIC (S) (I) | GNA11 (S) | MLLT3 (I) | PPP6C (S) | TET1 (I) |
| CSF3R (I) | GNAQ (S) | MRE11A(I) | PREX2 (S) | TGFBR2 (S) |
| CTCF (S) (I) | GNAS (S) | MSH2 (I) | PRKDC (S) (I) | TOP2A (I) |
| CTNNA1 (S) | H3F3C (S) | MSH3 (I) | PTCH1 (I) | TSC1 (I) |
| CYLD (I) | HIST1H3B (S) | MSH6 (S) | PTEN (S) (I) | XPO1 (S) |
| DDR2 (S) | HIST1H3C (S) | MST1 (S) | PTPN11 (S) | XRCC2 (I) |
| DIS3 (S) | HNF1A (I) | MYB (I) | PTPRD (S) | ZFHX3 (I) |
| DNMT1 (S) | HRAS (S) | MYC (S) | PTPRT (S) (I) | |
| DNMT3A (S) | IDH1 (S) | MYCN (S) (I) | QKI (I) | |

In another example, an indicator set can include genomic regions of viruses indicative of viral-associated cancers (“Viral”). For instance, viruses positively associated with cancer were identified in the CCGA Study using whole genome bisulfite sequencing. The panel generator 250 can determine an optimal number of target regions to be included in the detection panel in accordance with various embodiments described herein. Merely by way of example, a viral indicator set can include 10 sites in each of the following genomic regions: HPV16, HPV18, HBV, and EBV.

Other indicator sets are also possible.

### V. Disease Detection Panels

### V.A Assay Panels

Processing system 200 includes a panel generator 250 configured to generate a disease detection panel (“panel”) for determining a disease state, such as a presence or absence of a disease (“disease classification”) in a patient. The panel, in some cases, can also be used to determine a stage and/or a tissue of origin for the disease. Generally, the panel is applied to a sample (e.g., blood, tissue, etc.) obtained from the patient to determine a disease classification. For convenience, herein, example panels generated of the panel generator 250 will be configured to classify the presence of a cancer in a sample (“cancer presence”), but other diseases are also possible.

A panel includes a set of genomic regions. Each genomic region in the panel includes one or more sequences of nucleobases located at one or more particular sites on a chromosome (“coding regions”). The genomic regions can have one or more features whose variations are indicative of a disease state, such as a cancer presence or absence, a cancer stage and/or severity, and/or a cancer type (e.g., tissue of origin of a predicted cancer). As an example, a cancer detection panel can include genomic region CTNNB1, which is located at 3p22.1. A variation in a feature of CTNNB1 can be indicative of a cancer presence, and, more specifically, that cancer type is hepatobiliary cancer.

Each coding region in the panel is sequenced with one or more detection probes. A detection probe includes a complementary sequence of nucleobases corresponding to the nucleobases in the coding region. The detection probe, when applied to a sample, targets the nucleobase sequence in the coding region and pulls down nucleic acid fragments (i.e., test sequences). Test sequences include features, and variations in those features (“feature variation”) can indicate cancer presence. To illustrate, a feature can be a variation of indels at the coding region for a test sequence when compared to indels at that coding region in the population (e.g., healthy population).

The panel generator 250 generates panels which can be employed to determine cancer presence. To briefly illustrate, the panel generator 250 generates a panel comprising one or more detection probes for at least one genomic region. When applied to a sample, the detection probes generate test sequences for the coding region(s) associated with the genomic region(s). A processing system (e.g., system 200) identifies variants in the test sequences. The variant can be a single nucleobase variant (“SNV”), an insertion, or a deletion (the latter two collectively referred to as “indel”). The system 200 compares a feature of the variant against that same feature in the population (e.g., in a healthy population). A feature variation for that feature relative to the population can indicate cancer presence (e.g., presence of a cancer signal). Feature variations can be quantified as a feature value. For example, the system 200 can derive a feature value describing the maximum variant allele frequency (“maxVAF”) of a SNV. Accordingly, the system 200 can determine cancer presence in the sample based on the feature value. That is, if the maximum variant allele frequency of the SNV indicates cancer presence.

Other features, feature variations, and feature values are also possible. For example, feature values can quantify feature variations corresponding to at least one of a presence or absence of a variant, a mean allele frequency, a total number of small variants, and/or an allele frequency of true variants.

In some configurations, the system 200 can determine a likelihood of cancer presence based on feature values. For example, for each genomic region, a particular maxVAF for an SNV can correspond to a likelihood of a cancer presence. Accordingly, the system 200 can determine that the sample includes cancer presence if the determined likelihood is above a threshold likelihood.

### V.B Panel Size

The panel generator 250 generates panels having a panel size. The panel size is the total number of nucleobases of the genomic regions included in the panel. In some examples, each of the genomic regions has a maximum variant allele frequency for a single nucleotide variant of the genomic region, and at least some of the variant allele frequencies for the genomic regions occur in cancerous samples. Giving additional context, once the genomic regions for the panel are determined, the panel generator 250 can further determine the probe coverage of the panel (e.g., using probe generator 260). In some examples, the probe generator 260 tiles the probes to cover overlapping portions of each target genomic region included in the panel. For instance, the probes of the panel can be arranged pairwise such that each pair of probes overlaps each other with an overlapping sequence of, e.g., 60-nucleotides. Other lengths for the overlapping sequence are possible, such as 10-, 20-, 30-, 40-, 50-, 70-, 80-, 90-, 100-nucleotide overlap lengths and so on, and in some cases can depend upon a desired probe size described below. In such examples, the overall probe coverage size of the panel is much larger than the panel size itself. The probes of the panel can be applied to a sample to generate test sequences employed to determine cancer presence.

A probe included in a panel has a probe size, and the probe size is the number of nucleobases (or nucleotides, used interchangeably herein) in the probe. For example, a probe that includes the nucleobases [CAGGTCGAATTC] has a probe size of 12 nucleobases. Other probes having other probe sizes are also possible. For example, probes can have 40, 60, 80, 100, 120, 140, 160, 200 or some other number of nucleobases. In some examples, that number of nucleobases can include or otherwise be combined with an additional number of nucleobases serving as flanking regions with primer sequences. Such flanking regions can be located at the ends of the probes and have an additional 10, 20, 30, 40, 50, 60 or other number of nucleobases. For instance, a probe size of 120 bases plus 40 bases for flanking regions (e.g., 20-base flanking region at each end of a probe) yields an overall size of 160 nucleobases per probe. Typically, probes in a panel have the same probe size.

As used herein, a genomic region probed by a panel has an indicator size. The indicator size is the sum of the probe sizes for probes corresponding to that genomic region. To illustrate, a panel includes a first genomic region indicative of cancer presence. The first genomic region is sequenced by four probes having a probe size of 120 nucleobases. Thus, the indicator size for the genomic region is 480 nucleobases.

The total probe size of the panel, therefore, is the sum of the indicator sizes for all genomic regions included in a panel. To illustrate, a panel includes a first genomic region and a second genomic region. The first genomic region has an indicator size of 2.3 k nucleobases (or “kb”) and the second genomic region has an indicator size of 5.8 kb. Therefore, the total probe coverage size for the panel is 8.1 kb.

### V.D Panel Detection Capability

There are several metrics that quantify the disease detection capability of a panel. In an example, the panel generator 250 generates panels having a detection sensitivity and/or a detection specificity. Detection sensitivity is a quantification of a true positive rate for the panel, and detection specificity is a quantification of a true negative rate for the panel. Other metrics for quantifying the capability of the panel are also possible.

To illustrate, a system 200 employs a panel generated by panel generator 250 to determine cancer presence in 95 samples. The samples include 80 cancerous samples and 15 non-cancerous samples. The system 200 determines that 70 of the cancerous samples and 1 of the non-cancerous samples are indicative of cancer. The system 200 also determines that 10 of the cancerous samples and 14 of the non-cancerous samples are not indicative of cancer. Therefore, the detection sensitivity of the panel is 88% and the detection specificity of the panel is 93%.

### V.E Performance Metrics

The panel generator 250 can generate a panel based on a performance metric. Performance metrics can include, for example, panel size, panel detection capability, target disease (e.g., cancer), type of disease (e.g., throat cancer, liver cancer, etc.), and/or stage of disease (e.g., Stage I, Stage II, etc.), etc.

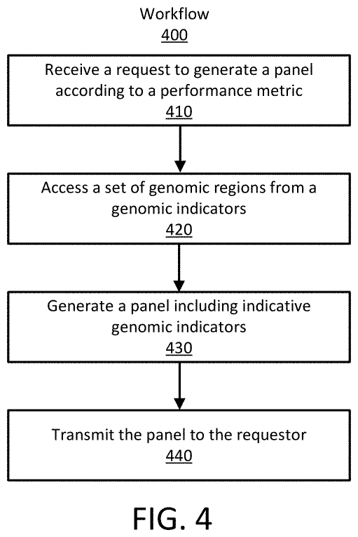

To illustrate, FIG. 4 shows an example workflow for generating a panel according to a performance metric according to an embodiment. The workflow 400 can be executed by the system 200 or another similar system. The workflow 400 can include additional or fewer steps, and the steps can be arranged in a different order.

The system 200 receives 410 a request to generate a panel that determines a disease classification (e.g., cancer). The request includes a performance metric defining how the panel should be designed. The panel generator 250 accesses 420 one or more indicator sets from the indicator database 290, each set including one or more genomic regions and its sequencing data. The panel generator 250 generates 430 a panel by selecting one or more of the accessed genomic regions whose variations can indicate a cancer presence. Determination of indicative genomic regions and their selection for the panel are described in greater detail below. The panel generator 250 transmits 440 the panel including the selected genomic regions to the requestor. In some examples, the panel generator 250 (e.g., via probe generator 260) determines or otherwise designs a set of probes that cover the selected genomic regions and transmits the probes and/or probe coverage to the requestor.

### VI. Classification Model

The panel generator 250 employs a classification model 270 to identify genomic regions to include in a panel. The classification model 270 identifies genomic regions by predicting the classification ability of panels including different combinations of identified genomic regions. The classification model 270 can include several different models, and each model can identify different genomic regions.

To generate a panel, the panel generator 250 accesses an indicator set including one or more genomic regions (e.g., from indicator database 290) and inputs them into the classification model 270. The panel generator 250 utilizes the classification model 270 to determine which of the accessed genomic regions can indicate a cancer presence (“indicators”), and selects the appropriate indicators for inclusion into the panel. Each of the various models in the classification model 270 can determine indicators to include in the panel in a different manner. For example, the related gene model 272 can determine that a genomic region whose feature variation is associated with cancer presence should be included in the panel as a related indicator. In another example, the viral region model 280 can determine that genomic regions associated with viruses associated with cancers should be included in the panel as viral indicators. The various models are described in more detail herein.

Several other configurations of a classification model 270 are also possible. In a configuration, the panel generator 250 employs the classification model 270 to determine indicators for a panel according to one or more performance metrics. For example, the panel generator 250 can generate a panel having the highest detection sensitivity while having a panel size less than a threshold panel size. In another example, the panel generator 250 can generate a panel having the smallest panel size while having a detection sensitivity above a threshold sensitivity.

In another configuration, the panel generator 250 can generate panels having increased detection capability when the classification model 270 determines indicators based on more than one feature. As an example, a classification model 270 can determine indicators based on feature variations for both SNVs and indels.

### VI.A Example Classification Model Performance

The detection capability of a panel depends on the configuration of the classification model 270. A receiver operating characteristic curve plot (“ROC plot”) visualizes the detection capability of a panel. In a ROC plot, the x-axis is the false positive rate and the y-axis is the true positive rate. The false positive rate is 1 less the specificity and the true positive rate is the sensitivity.

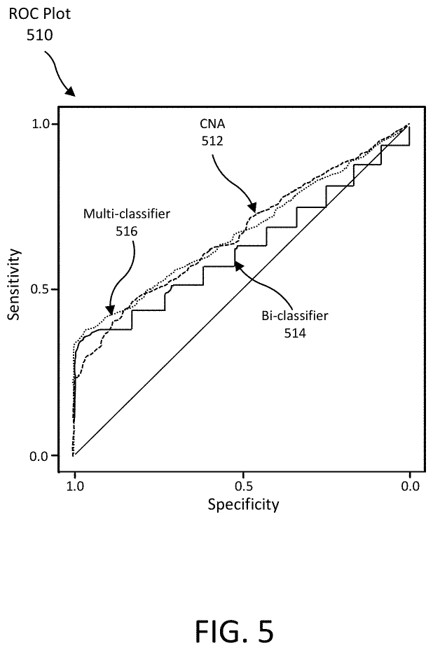

FIG. 5 illustrates a ROC plot showing performance of three classifiers based on a panel that includes a large set of genomic regions (approximately 2 Mb) that were not identified or selected in the manners described herein. The ROC plot 510 includes three curves showing the cancer/non-cancer detection capability of the three example classification models 270. The first curve shows the detection capability of the panel generated by a classification model configured to analyze feature variations in copy number aberrations (“CNA”) to determine cancer presence (CNA 512). The second curve shows the detection capability of the panel generated by a classification model configured to analyze feature variations in SNVs and indels to determine cancer presence (Bi-classifier 514). The third curve shows the detection capability of the panel generated by a classifier configured to analyze feature variations in SNVs, indels, and CNAs (Multi-classifier 516). Table 7 gives a comparison of the detection capability of the three models shown in FIG. 5.

TABLE 7
Detection capability of example classifiers
on large set of genomic regions

| Classifier | 95% Specificity | 98% Specificity | 99% Specificity |
|---|---|---|---|
| SNV/INDEL | 0.3697 | 0.3479 | 0.3348 |
| CNA | 0.3053 | 0.2541 | 0.2334 |
| MULTI | 0.3860 | 0.3675 | 0.3490 |

### VII. Related Indicators

As described above, the classification model 270 includes a related gene model 272 (“related model 272”). The related model 272 determines which genomic regions in an indicator set are related to cancer presence. To quantify relations between genomic regions and cancer presence, the panel generator 250 determines a model coefficient for each of the genomic regions. For the related model 272, a model coefficient quantifies a feature value's indicativeness for cancer presence for a genomic region (“sensitivity coefficient”). For example, a sensitivity coefficient of 0.05 indicates a low likelihood that a derived feature value for a genomic region indicates cancer presence, while a sensitivity coefficient of 0.55 indicates a high likelihood that a feature value for a genomic region indicates cancer presence.

To provide context, consider an accessed indicator set including a genomic region. The genomic region is associated with cancerous and non-cancerous sequencing data in the indicator set. The panel generator 250 derives and analyzes feature values for the sequencing data. For example, the panel generator 250 determines the maxVAF for SNVs in the accessed sequencing data. In this case, if variation in the maxVAF for SNVs in the sequencing data is indicative of cancer presence, the panel generator 250 determines the genomic region has a high sensitivity coefficient (e.g., 0.60). Conversely, if variation in the maxVAF for SNVs in the sequencing data is not indicative of a cancer presence, the genomic region has a low sensitivity coefficient (e.g., 0.06).

There are several methods to determine model coefficients. In an example, the panel generator 250 employs the related model 272 to perform a L2 penalized logistic regression on accessed sequencing data. In this case, the model coefficient (e.g., sensitivity coefficient) is the regression coefficient determined for each genomic region. In other examples, the classification model 270 can perform L1 penalized logistic regression, elastic net classifier logistic regression support vector machines (SVMs), Naïve Bayes, and random forests to determine model coefficients.

The panel generator 250 employs the classification model 270 to rank accessed genomic regions based on their determined model coefficients. The panel generator 250 then selects genomic regions for the panel as related indicators. Ranking and selecting related indicators is described in more detail below.

### VII.A Related Model Performance

The regression-based models described herein (e.g., related model 272) have greater detection capability than those found for the large set of genomic regions. To illustrate, Table 8 compares the detection capability of a panel (e.g., a reduced, optimized panel) generated using a regression-based classification model 270 against a classification model from the large set of genomic regions shown above at Table 7. More specifically, the table compares the detection capabilities for panels configured for analyzing feature variations for both SNVs and indels. Further, the table compares the detection capability of three different logistic regression based classification models against the that of the large set of genomic regions. As shown in the table, log-reg-l2 is a L2 logistic regression classifier, log-reg-L1 is a L1 logistic regression classifier, and log-reg-en is an elastic net logistic regression classifier. As shown, classifier performance based on the reduced panel using L2 or elastic net logistic regression improved over that of the large set of genomic regions across the 95%, 98%, and 99% specificities, while classifier performance of the reduced panel using L1 logistic regression generally achieved similar performance or otherwise reproduced/maintained the performance of the large set classifier across the specificities.

TABLE 8
Classification model comparison

| SNV/Indel | | | |
|---|---|---|---|
| Classifier | 95% Specificity | 98% Specificity | 99% Specificity |
| large set | 0.3697 | 0.3479 | 0.3348 |
| classifier | | | |
| log-reg-L2 | 0.3944 | 0.3745 | 0.3587 |
| log-reg-L1 | 0.3676 | 0.3440 | 0.3306 |
| log-reg-en | 0.3944 | 0.3685 | 0.3508 |

### VII.B Mono-Classifiers and Bi-Classifiers

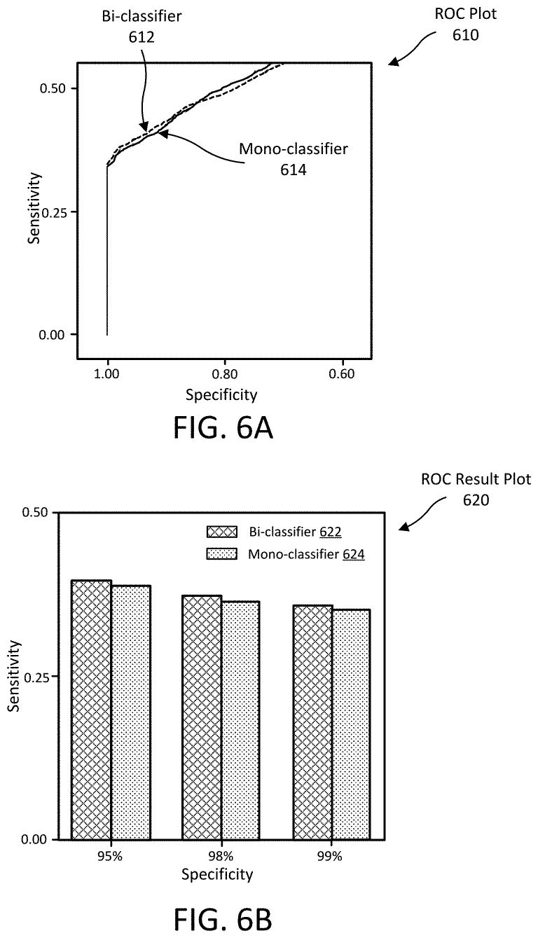

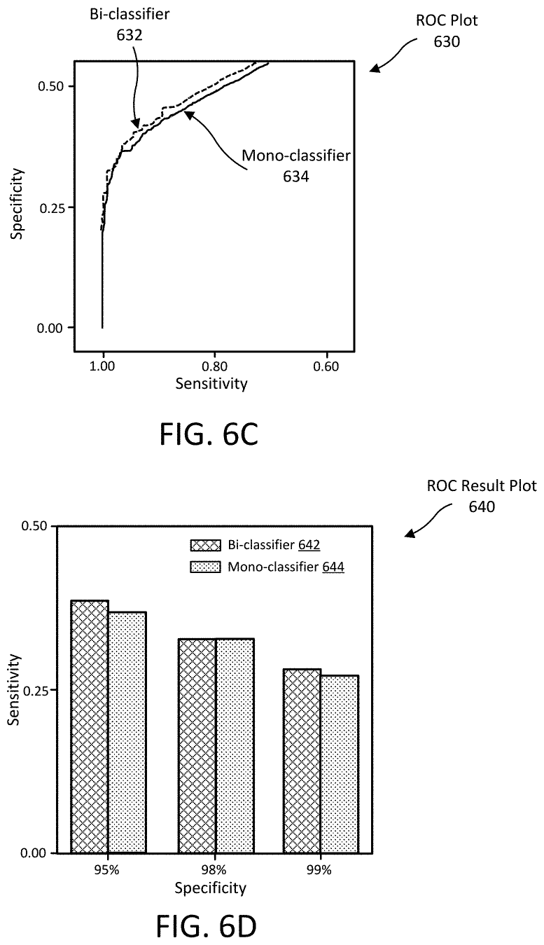

The panel generator 250 can employ a classification model 270 to generate panels by analyzing one or more derived feature values for a genomic region. Generally, panels generated based on two feature values (i.e., based on both SNVs and indels) achieved similar detection capability as those generated based on a single feature value (e.g., SNVs only). To illustrate, FIG. 6A-6D demonstrate the detection capability of panels generated by a panel generator 250 employing a classification model analyzing feature values for SNVs and indels (“bi-classifier”), and a classification model analyzing features values for SNVs only (“mono-classifier”). In FIG. 6A-6D, the classifiers are applied to samples including both low-signal and high-signal cancers.

FIG. 6A illustrates a ROC plot for panels generated by a bi-classifier and mono-classifier that are applied to training data including both low-signal and high-signal cancers, according to some embodiments. The bi-classifier 612 comprises a L2 logistic regression classifier with SNV and indels as features, while the mono-classifier 614 is a L2 logistic regression classifier on SNVs only. As shown in the ROC plot 610, the bi-classifier 612 has slightly better detection capabilities than the mono-classifier 614 at high detection sensitivities, but the performance is generally the same.

FIG. 6B illustrates a ROC result plot for the ROC plot in FIG. 6A according to some embodiments. In a ROC result plot, the x-axis is the specificity and the y-axis is the sensitivity. A ROC result plot compares the sensitivity of the bi-classifier to the mono-classifier at different specificities. As shown in the ROC result plot 620, the bi-classifier 622 has slightly higher sensitivity for specificities relative to the mono classifier 624, but still the performance is generally the same. In other words, using only SNVs for a panel design in accordance with the methods described herein would result in only a minimal loss of clinical sensitivity (e.g., 1-2%) while allowing for a simpler and more cost-effective panel.

FIG. 6C illustrates a ROC plot for panels generated by a bi-classifier and mono classifier that are applied to test data according to some embodiments. For example, subsequent to training the bi-classifier and mono-classifier on the training data as in FIGS. 6A-6B, the trained classifiers can perform classification on a set of test data. As in FIGS. 6A-6B, the bi-classifier 632 comprises a L2 logistic regression classifier with SNV and indels as features, while the mono-classifier 634 is a L2 logistic regression classifier on SNVs only. As shown in the ROC plot 630, the bi-classifier 632, generally, has minimally better detection capabilities than the mono-classifier 634, resulting in similar classification performance.

FIG. 6D illustrates a ROC result plot for the ROC plot of FIG. 6C according to some embodiments. As shown in the ROC result plot 640, the bi-classifier 642 has minimally higher sensitivity at 95% and 99% specificities relative to the mono classifier 644 and the same sensitivity at 98% specificity as the mono-classifier 644. In other words, classification on the test data confirms that using only SNVs for a panel design as described herein would achieve similar performance as a panel designed for both SNVs and indels, while also providing a more simple panel.

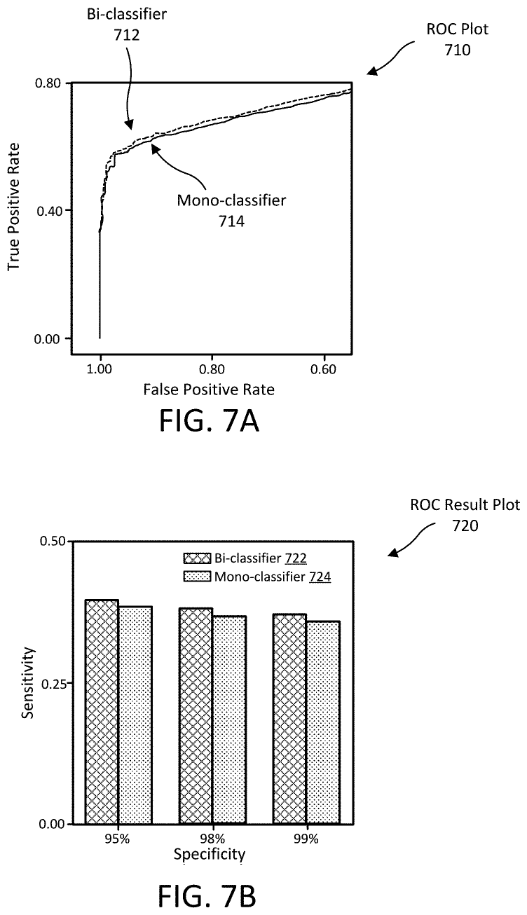

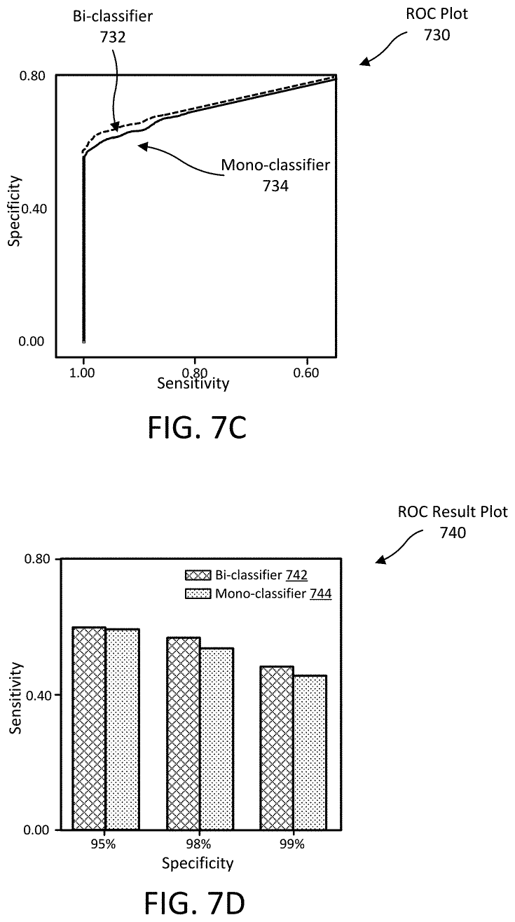

FIGS. 7A-7D further illustrate the increase in detection capability of bi-classifiers relative to mono-classifiers for high signal cancers only. Specifically, in FIGS. 7A-7D, the panels are applied to samples including only high-signal cancers, rather than both high signal and lower-signal cancers as in FIGS. 6A-6D. Both classifiers shown in FIGS. 7A-7D comprise L2 logistic regression.

FIG. 7A illustrates a ROC plot for panels generated by a bi-classifier and mono-classifier that are applied to training samples according to some embodiments. As shown in the ROC plot 710, the bi-classifier 712 has minimally better detection capabilities than the mono-classifier 714 at high detection sensitivities. Therefore, using only SNVs for a panel design for high signal cancers in accordance with the methods described herein would result in only a minimal loss of clinical sensitivity while allowing for a simpler and more cost-effective panel.

FIG. 7B illustrates a ROC result plot for the ROC plot of FIG. 7A according to some embodiments. As shown in the ROC result plot 720, the bi-classifier 722 has minimally higher sensitivity for all specificities relative to the mono classifier 724. Therefore, the bi-classifier 722 and mono classifier 724 can be considered to achieve similar classification performance on high signal cancers.

Table 9 compares the results of the panels in FIGS. 7A and 7B.

TABLE 9
Comparison between classifier types for training data

| Log-reg-L2 | | | |
|---|---|---|---|
| Classifier | 95% Specificity | 98% Specificity | 99% Specificity |
| Bi-Class. | 0.6330 | 0.6116 | 0.5937 |
| (SNV + Indel) | | | |
| Mono-class. | 0.6124 | 0.5881 | 0.5736 |
| (SNV) | | | |

FIG. 7C illustrates a ROC plot for panels generated by a bi-classifier and mono classifier that are applied to high signal cancer test samples according to some embodiments. For example, subsequent to training the bi-classifier and mono-classifier on the high signal cancer training data as in FIGS. 7A-7B, the trained classifiers can perform classification on a set of high signal cancer test data. As shown in the ROC plot 730, the bi-classifier 732 has minimally better detection capabilities than the mono-classifier 734 at high detection sensitivities.

FIG. 7D illustrates a ROC results plot of the ROC plot in FIG. 7C according to some embodiments. As shown in the ROC results plot 740, the bi-classifier 742 has minimally higher sensitivity for all specificities relative to the mono-classifier 744. Therefore, as classification on the test data further shows, using only SNVs for a panel design for high signal cancers in accordance with the methods described herein would result in only a minimal loss of clinical sensitivity while allowing for a simpler and more cost-effective panel.

Table 10 compares the results of the panels in FIGS. 7C and 7D.

TABLE 10
Comparison between classifier types for real data

| Log-reg-L2 | | | |
|---|---|---|---|
| Classifier | 95% Specificity | 98% Specificity | 99% Specificity |
| Bi-Class. | 0.6007 | 0.5714 | 0.4835 |
| (SNV + Indel) | | | |
| Mono-class. | 0.5934 | 0.5385 | 0.4578 |
| (SNV) | | | |

### VIII. Ranking Genomic Regions

As described above, the panel generator 250 generates a panel by applying a classification model 270 to accessed genomic regions. The classification model 270 includes a related model 272 that derives feature values for each of the accessed indicators. The related model 272 then determines model coefficients for the genomic regions and ranks the genomic regions based on their model coefficients. Here, the model coefficient is the regression coefficient of a regression based classifier, but could be another quantification of a genomic region's indicativeness for cancer presence.

It is noted that one of more models of the classification prediction model 270 can include regression-based classifiers and/or other models for ranking genomic regions or otherwise selecting genomic regions to be included in a panel design. For instance, the related model 272 can comprise a logistic regression classifier trained on a set of training data, such as a set of training data comprising high signal cancers and/or other cancers as discussed above in FIGS. 6A-6D and 7A-7D. Further, the related model 272 can comprise a mono-classifier that uses SNVs only for a SNV-only panel design, or a bi-classifier that uses SNVs and indels for a SNV and indel panel design. As discussed above, in some cases, SNV-only based classification for an SNV-only panel can be preferred over a combined SNV and indel approach when similar classification performance can be expected or otherwise achieved. Still further, in some examples, one or more of the models for ranking or selecting genomic regions can include models or methodologies for customizing or curating genomic regions from various sources, such as databases and/or literature. It is noted that the classification prediction model 270 can include any combination of such classification models and/or customization techniques, as discussed further below.

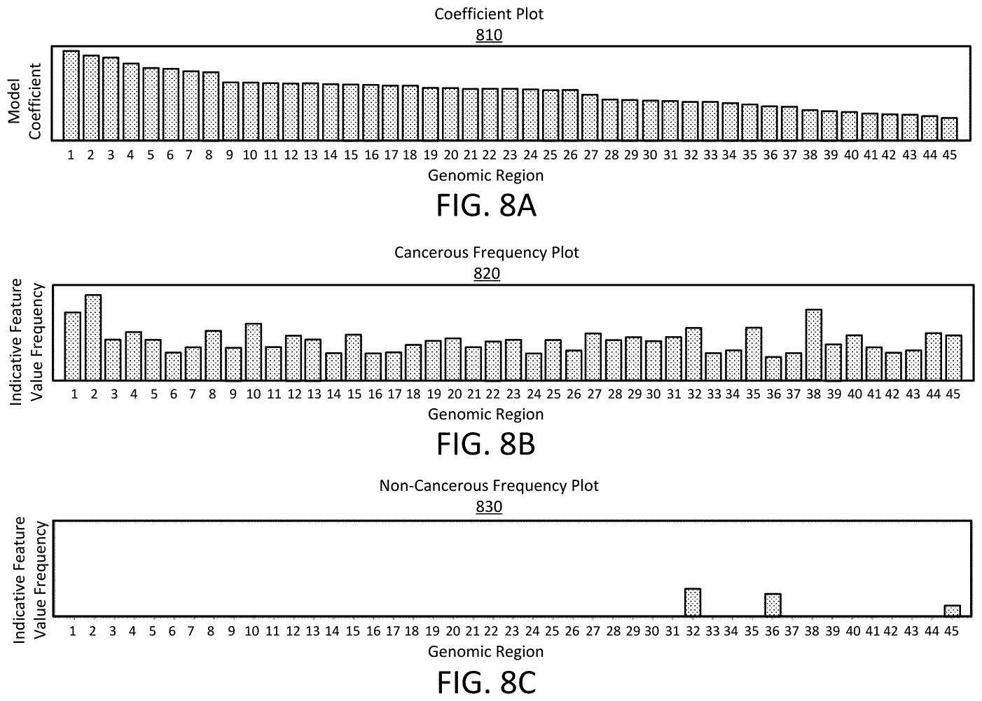

FIGS. 8A-8C, 9A-9C, and 10 illustrate model coefficients determined by a panel generator 250 applying a related model 272 to an indicator set. The indicator set can be, for example, the CCGA indicator set that includes both solid and/or liquid sequencing data. The related model 272 can be a regression based classifier, such as a L2 logistic regression classifier trained on a set of training data (e.g., high signal cancers only training data, or high and low signal cancers training data).

### VIII.A Solid Cancers

FIG. 8A illustrates a coefficient plot for 45 genes related to high signal cancers (e.g., solid cancers) according to some embodiments. A coefficient plot illustrates model coefficients for a number of genomic regions. That is, each bar on the x-axis represents a different gene or genomic region, and the height of the bar along the y-axis is a quantification of the genomic region's model coefficient (in arbitrary units).

In the coefficient plot 810, genomic regions are ranked according to their determined model coefficients. That is, the genomic regions are ranked according to their feature values indicating or being informative of a cancer presence. Here, the genomic regions correspond to genes related to solid cancers and are listed in Table 11 below. Therefore, genomic regions on the left side of the coefficient plot 810 are more indicative of solid cancer presence than genomic regions on the right side of the coefficient plot 810.

FIG. 8B illustrates a cancerous frequency plot for solid cancers according to one embodiment. A cancerous frequency plot illustrates an indicative feature value frequency for genomic regions in samples having a cancer presence. That is, each bar on the x-axis represents a different genomic region, and the height of the bar on the y-axis is a quantification of how often a feature value in that genomic region indicates a cancerous sample. Further, the genomic region at each position on the x-axis is the same genomic region in the corresponding position in the coefficient plot of FIG. 8A. For example, genomic region 1 in FIG. 8A is the same as genomic region 1 in FIG. 8B, etc.

In the illustrated cancerous frequency plot 820, the feature indicative of cancer is the maximum variant allele frequency for an SNV of the genomic region. Therefore, the indicative feature value frequency is a quantification of how often an indicative maximum variant allele frequency occurs in samples having a solid cancer presence. Notably, indicative feature value frequencies for genomic regions are not similarly ranked to their corresponding model coefficients. This indicates that a high indicative feature variation frequency does not necessarily correspond to that genomic region being highly indicative of cancer presence.

FIG. 8C illustrates a non-cancerous frequency plot for solid cancers according to one embodiment. A non-cancerous frequency plot illustrates an indicative feature value frequency for genomic regions in non-cancerous samples. Here, the genomic region at each position on the x-axis is the same genomic region in the corresponding positions in FIGS. 8A and 8B.

In the non-cancerous frequency plot 830, the indicative feature value frequency is a quantification of how often an indicative maximum variant allele frequency occurs in non-cancerous samples. The frequencies in the non-cancerous samples are much lower than the frequencies in cancerous samples, indicating that the illustrated indicators have a high specificity.

### VIII.B Liquid Cancers

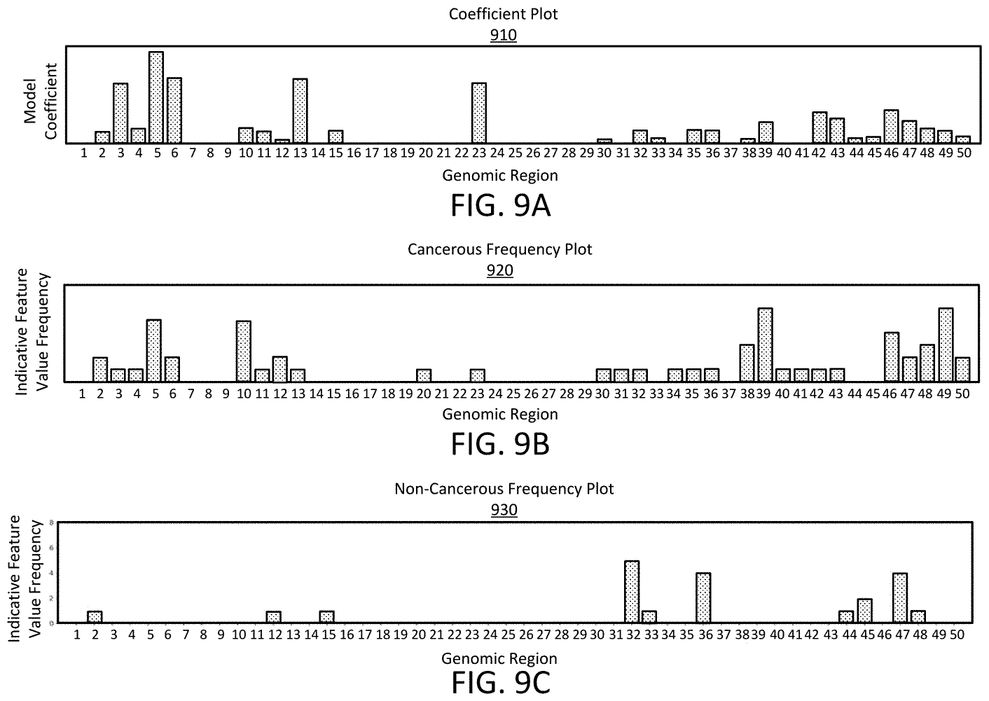

FIGS. 9A-9C illustrate plots similar to FIGS. 8A-8C, except the model coefficients and feature variation frequencies are derived from a regression classifier trained on liquid cancer samples. Additionally, FIGS. 9A-9C include several supplementary genomic regions (i.e., genomic regions 46-50). The genomic region at each position on the x-axes in FIGS. 9A-9C is the same genomic region in the corresponding positions in FIGS. 8A-8C.

FIG. 9A illustrates a coefficient plot for the genomic regions when applied for detection of liquid cancers according to some embodiments. In coefficient plot 910, the genomic regions are listed along the x-axis in order of their ranking for indicating solid cancer presence. However, the genomic regions are not appropriately ranked for liquid cancer detection because the model coefficients for liquid cancer are dissimilar to the model coefficients for solid cancer. Additionally, the supplementary genomic regions have higher model coefficients than many of the original genomic regions. This indicates that the panel generator 250 can select genomic regions for the panel based on the type of cancer it will be probing.

FIG. 9B illustrates a cancerous frequency plot for liquid cancers according to some embodiments. In the cancerous frequency plot 920, the indicative feature value frequency is a quantification of how often an indicative maximum variant allele frequency occurs in cancerous samples. The genomic region at each position on the x-axis is the same genomic region in the corresponding positions in FIGS. 8A-8C. Similar to FIG. 8B, the feature variation frequency does not correspond to the ranking of the genomic region.

FIG. 9C illustrates a non-cancerous frequency plot for liquid cancers according to some embodiments. In the non-cancerous frequency plot 930, the indicative feature value frequency is a quantification of how often an indicative maximum variant allele frequency occurs in non-cancerous samples. Similar to FIG. 8C, the frequency variation in non-cancerous samples is much lower than those in cancerous samples.

VIII.C Solid Vs. Liquid Cancers

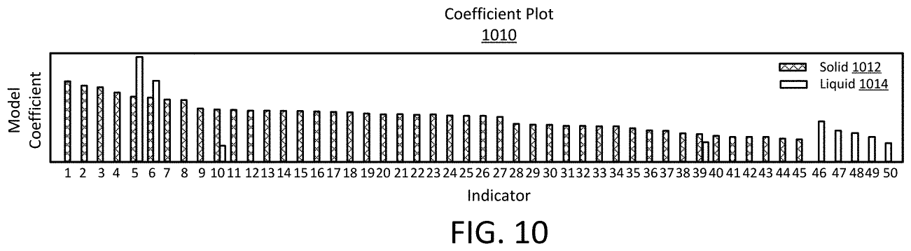

FIG. 10 illustrates a coefficient plot for solid and liquid cancers according to some embodiments. The coefficient plot 1010 illustrates differences between model coefficients of genomic regions for solid and liquid cancers. In the coefficient plot 1010 the filled bars represent the model coefficient solid cancer 1012, while the unfilled bars represent the model coefficient for liquid cancer 1014. The genomic region at each position on the x-axis is the same genomic region in the corresponding positions in FIGS. 9A-9C. As shown, model coefficients for genomic regions 5, 6, 10, and 39 are indicative of a cancer presence for both solid and liquid cancers. Model coefficients in genomic regions 1-45 are, generally, indicative of solid cancer presence, while model coefficients in genomic regions 46-50 are, generally, indicative of liquid cancer presence.

### IX. Selecting Indicators

As described above, the panel generator 250 generates a panel by applying a classification model 270 to accessed genomic regions. The classification model 270 determines and ranks model coefficients for each genomic region. The panel generator 250 then selects genomic regions for the panel as indicators based on their ranked model coefficients.

The panel generator 250 can select indicators in several ways. In a first configuration, the panel generator 250 determines model coefficients from feature values and ranks those coefficients in a single iteration. The panel generator 250 can then select genomic regions for the panel based on the single iteration's ranking. The classification model 270 can also be applied to different indicator sets and selected in a similar manner for each indicator set.

In another configuration, the panel generator 250 can determine and rank model coefficients after each genomic region is selected for the panel. For example, after selecting the genomic region with the highest ranked coefficient after a first iteration, the panel generator 250 model can apply the classification model 270 to the remaining indicators to derive features and rank model coefficients in a second iteration. The panel generator can then select genomic regions based on model coefficients determined in the second iteration. The iterative selection process can continue as needed and can include different indicator sets.

Additionally, there are several design aspects to consider when deciding how to configure the panel generator 250 to select indicators. Some classification models select as many indicators as possible for a panel, believing each additional indicator increases the detection capability of that panel. However, the detection capability of a panel does not necessarily increase with each additional indicator, as described below. Further, selecting additional indicators for a panel increases the complexity and cost of that panel. Therefore, the panel generator 250 can be configured to select indicators based on a performance metric. Some performance metrics include detection capability (e.g., classification sensitivity, classification accuracy), panel size, panel target (e.g., solid, liquid, etc.), and/or any combination thereof, as described above.

### IX.A Detection Capability

The panel generator 250 can generate a panel with an optimized detection capability. One performance metric for measuring detection capability is, for example, panel sensitivity at 95% specificity (“detection capability metric”), but other performance metrics are also possible. Accordingly, in this example, the panel generator 250 continually selects genomic regions as related indicators until the performance metric decreases, tapers off, and/or plateaus with addition of another genomic region or related indicator. The related indicators can be iteratively selected, with each iteration selecting the indicator with the highest determined model coefficient.

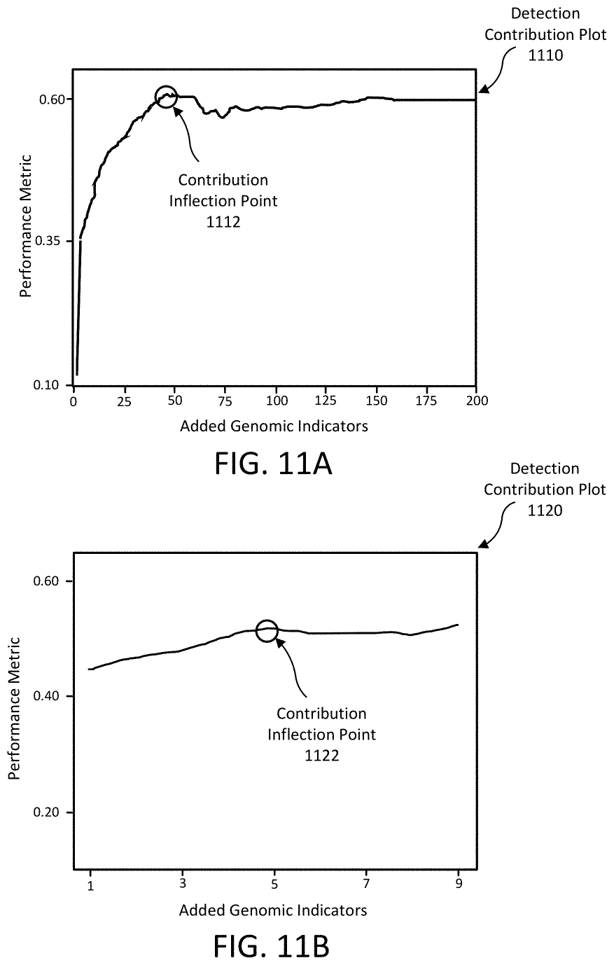

To illustrate, FIG. 11A shows a detection contribution plot for solid cancers according to some embodiments. In the detection contribution plot 1110, the x-axis represents genomic regions added to a panel, and the y-axis illustrates the detection capability metric for that panel. Here, the performance metric is sensitivity at a given specificity. The genomic regions are added to the panel in ranked order according to their model coefficient for solid cancers. As shown, adding genomic regions to the panel increases the detection capability metric until a contribution inflection point 1112. At the contribution inflection point 1112, adding additional genomic regions decreases the detection capability metric. In the illustrated example, the contribution inflection point 1112 occurs at 45 genomic regions, after which the detection capability metric decreases. Accordingly, the panel generator 250 can select the first 45 genomic regions (e.g., out of a large set of 200 genomic regions) as related indicators for the panel. Table 11 gives, for example, 45 related indicators selected for the panel for determining solid cancer presence. The table shows their name, size, and location on the genome.

TABLE 11
Related classifiers selected for solid cancers

| Num. | Gene Name | Size (bp) | Locus |
|---|---|---|---|
| 1 | KRAS | 687 | 12p12.1 |
| 2 | TP53 | 1,263 | 17p13.1 |
| 3 | ERBB2 | 3,796 | 17q12 |
| 4 | EPHB1 | 2,955 | 3q22.2 |
| 5 | NRAS | 570 | 1p13.2 |
| 6 | ACVR1B | 1,641 | 12q13.13 |
| 7 | TP63 | 2,256 | 3q28 |
| 8 | KEAP1 | 1,875 | 19p13.2 |
| 9 | CDK12 | 4,473 | 17q12 |
| 10 | KMT2D | 16,614 | 12q13.12 |
| 11 | DICER1 | 5,769 | 14q32.13 |
| 12 | TET2 | 6,009 | 4q24 |
| 13 | LATS2 | 3,267 | 13q12.11 |
| 14 | ETV5 | 1,533 | 3q27.2 |
| 15 | GRIN2A | 4,395 | 16p13.2 |
| 16 | EPHA7 | 2,997 | 6q16.1 |
| 17 | ASXL2 | 4,308 | 2p23.3 |
| 18 | RET | 3,345 | 10q11.21 |
| 19 | CHD2 | 5,487 | 15q26.1 |
| 20 | RB1 | 2,787 | 13q14.2 |
| 21 | CDH1 | 2,649 | 16q22.1 |
| 22 | PDGFRA | 3,473 | 4q12 |
| 23 | BRCA2 | 10,257 | 13q13.1 |
| 24 | TFRC | 2,283 | 3q29 |
| 25 | ALK | 4,863 | 2p23.2 |
| 26 | KDM5A | 5,073 | 12p13.33 |
| 27 | SMAD4 | 1,659 | 18q21.2 |
| 28 | ATR | 7,935 | 3q23 |
| 29 | NOTCH1 | 7,668 | 9q34.3 |
| 30 | NRG1 | 3,616 | 8p12 |
| 31 | CTNNB1 | 2,346 | 3p22.1 |
| 32 | KMT2C | 14,736 | 7q36.1 |
| 33 | SNCAIP | 3,051 | 5q23.2 |
| 34 | MTOR | 7,650 | 1p36.22 |
| 35 | PIK3CA | 3,207 | 2q23.32 |
| 36 | SF3B1 | 3,935 | 2q33.1 |
| 37 | NBN | 2,265 | 8q21.3 |
| 38 | LRP1B | 13,800 | 2q21.1 |
| 39 | TRFRSF14 | 852 | 1p36.32 |
| 40 | ARID1A | 6,858 | 1p36.11 |
| 41 | INPP4A | 3,115 | 2q11.2 |
| 42 | ETS1 | 1,540 | 11q24.3 |
| 43 | KAT6A | 6,015 | 8p11.21 |
| 44 | FBXW7 | 2,532 | 4q31.3 |
| 45 | MGA | 9,198 | 15q15 |

FIG. 11B shows a detection contribution plot for liquid cancers according to some embodiments. In the detection contribution plot 1120, the x-axis represents genomic regions added to a panel, and the y-axis illustrates the performance metric for that panel. Here, the performance metric is sensitivity at a given specificity. The genomic regions are added to the panel in ranked order according to their model coefficient for liquid cancers. In the illustrated example, the contribution inflection point 1122 is 5 genomic regions, after which the performance metric generally plateaus. Accordingly, the panel generator 250 can select the first 5 genomic regions (e.g., out of a larger set of 9 genomic regions) as related indicators for the panel. Table 12 gives, for example, 5 related indicators selected for the panel for determining liquid cancer presence. The table shows their name, size, and location on the genome.

TABLE 12
Related classifiers for liquid cancers

| Num. | Gene Name | Size (bp) | Position |
|---|---|---|---|
| 1 | MYD88 | 954 | 3p22.2 |
| 2 | CBL | 2,721 | 11q23.3 |
| 3 | BRAF | 2,301 | 7q34 |
| 4 | CREBBP | 7,329 | 16p13.3 |
| 5 | APC | 8,697 | 5q22.2 |

### IX.B Panel Size

The panel generator 250 can select ranked indicators to generate a panel with a panel size less than a threshold panel size. For example, the panel generator 250 can be configured to generate a panel less than 500 kb. The threshold panel size can be a configuration of the panel generator 250, a designation by a system 200 administrator, or received from a user of the system 200.

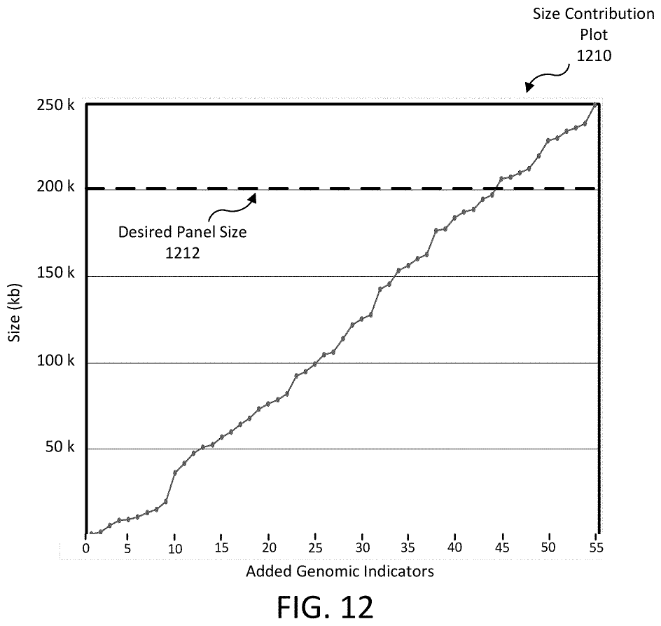

To illustrate, FIG. 12 shows a size contribution plot for solid cancers according to some embodiments. In the size contribution plot 1210, the x-axis represents the number of ranked genomic regions added to the panel, and the y-axis illustrates the panel size for the panel. A dashed horizontal line 1212 indicates a desired threshold panel size of 200 kb. As shown, adding genomic regions to the panel increases the panel size, and the 45^{th} added indicator increases the panel size above the threshold panel size. Accordingly, the selected panel includes the first 44 genomic regions.

### X. Additional Indicators

As described above, the panel generator 250 employs a classification model 270 to determine genomic regions to include as related indicators in a panel. As described hereto, the classification model selected genomic regions for the panel according to a related gene model 272. However, in some circumstances, the related gene model 272 may not identify some genomic regions that can increase the detection capability of the panel due its configuration. Accordingly, the classification model 270 can employ one or more additional models to identify and select additional genomic regions as indicators the panel. Some additional models, for example, a region coverage model 274, a cancer type model 276, a hotspot region model 278, and a viral region model 280, as described below.

### X.A Coverage Indicators

As described above, the panel generator 250 can access an indicator set including genomic regions from an indicator database 280. The panel generator 250 trains, for example, a related model 272 to generate a panel using identified indicators from the indicator set. However, in some cases, the indicator set is not suitable for training a related model 272. In these instances, the panel generator 250 can apply a different model to select additional genomic regions for the panel as coverage indicators that improve panel coverage. Coverage is a quantification of how many samples in the indicator set are identified by genomic regions included in a panel. Coverage is not a quantification of sensitivity.

To illustrate, consider an indicator set including genomic regions obtained from only cancerous samples. In this case, the panel generator 250 cannot train related model 272 because the indicator set includes genomic regions determined from cancerous samples, but lacks control data obtained from non-cancerous samples. Accordingly, the panel generator 250 can apply a region coverage model (“coverage model 274”) to determine coverage indicators to include in the panel.

A coverage model 274, in a manner similar to the related model 270, identifies a model coefficient for each genomic region in an indicator set. In this example, the model coefficient is a measure of how many additional samples (e.g., patient samples in the training and/or test sets) are identified when adding the genomic region to the panel (“coverage coefficient”). The panel generator 250 then ranks determined coverage coefficients, and, subsequently, selects genomic regions from the ranked list for inclusion into the panel as coverage indicators. The panel generator 250 can select the coverage indicators in their ranked order, by some other metric, or not at all.

For instance, in some examples, the coverage model 274 uses a greedy algorithm to add genes to the panel until performance (e.g., sensitivity) plateaus. For example, an initial panel can include top 50 genes selected by the related gene model 272 as described above. In some cases, additional data sets such as TCGA data can be used to identify additional genes to be included in the panel. In that case, performance (e.g., sensitivity) of the panel can be evaluated on the TCGA data, whereby the coverage model 274 identifies additional genes that further increase sensitivity of the panel in addition to the initial 50 genes. For instance, for an SNV panel design, the coverage model 274 can evaluate high signal cancers and liquid cancers from TCGA SNV data and subsequently use the greedy algorithm of adding genes to the panel until the sensitivity plateaus and/or a desired panel size is reached. In doing so, the coverage model 274 can rank genes in the TCGA data by frequency of somatic mutations per patient and/or by frequency normalized by the coding region length, and then examine how many additional patients (e.g., samples) can be captured or otherwise covered by adding TCGA genes. In some cases, the genomic regions identified by the coverage model 274 are considered candidate genes (e.g., TCGA genes), which can then be manually curated for addition to the panel by cross-checking with other databases, such as by observing mutation profiles on the GDC cancer portal and literature, in addition and/or alternative to evaluating their contribution to performance.

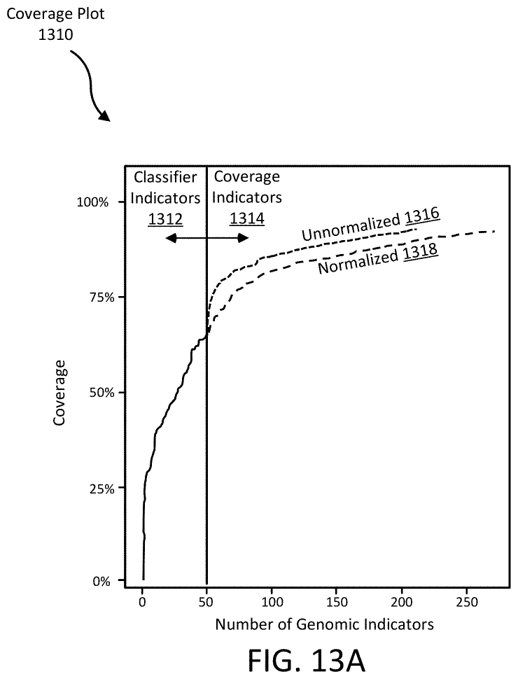

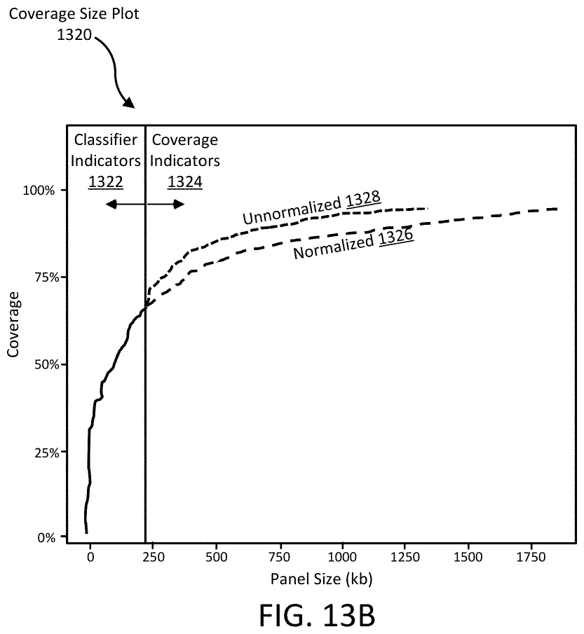

To illustrate, FIG. 13A shows a coverage plot according to some embodiments. A coverage plot shows the coverage of a panel applied with an accessed indicator set (e.g., TCGA indicator set). In the coverage plot 1310, the x-axis indicates the number of genomic regions selected for the panel, and the y-axis indicates the coverage (e.g., number of patient samples covered) of the panel. In this example, the first 50 genomic regions are related indicators 1312 selected according to the related model 272. The remaining genomic regions are coverage indicators 1314 from the TCGA genomic region indicator set selected according to the coverage model 274.

The coverage plot 1310 includes two lines depicting coverage of the coverage indicators: (i) a first line showing coverage as the number of indicators in the panel increases (e.g., unnormalized 1316), and (ii) a second line showing coverage as the number of indicators in the panel increases, normalized by coding region length (e.g., normalized 1318). In either case, the coverage plot 1310 shows asymptotic growth towards full coverage as the number of genomic regions in the panel is increased. The panel generator 250 can select any of the coverage indicators for the panel, in some cases depending on remaining space on the panel and/or desired size of the panel. For example, the panel generator 250 can select three coverage indicators for the panel. Table 13: indicates the name, size, and position, of the three coverage indicators selected for the panel.

TABLE 13
Coverage indicators selected for panel

| Num. | Gene Name | Size (bp) | Position |
|---|---|---|---|
| 1 | CDH10 | 2,367 | 5p14.2 |
| 2 | CSMD3 | 11,182 | 8q23.3 |
| 3 | NFE2L2 | 1,818 | 2q31.2 |

FIG. 13B shows a coverage size plot according to some embodiments. The coverage size plot 1320 conveys the information in FIG. 13A in a different manner. Here, the x-axis indicates the panel size, and the y-axis indicates coverage of the panel. Here, increase in panel size stems from adding genomic regions to the panel according to their respective models. The added genomic regions occur in the same order as coverage plot 1310 of FIG. 13A.

In the coverage size plot 1320, the first 240 kb of the panel size result from indicators selected according to the related model 272 (related indicators 1322), and the additional bases in the panel size are from indicators selected according to the coverage model 274 (coverage indicators 1324). Again, the coverage plot 1320 includes two lines: (i) a first line showing increasing coverage with increasing panel size (unnormalized 1328), and (ii) a second line showing increasing coverage with increasing panel size, but normalized by the coding region length of the added indicator (normalized 1326).

### X.B Cancer Type Indicators

As described above, the panel generator 250 accesses an indicator set and ranks indicative genomic regions according to their model coefficients. To this point, a model coefficient has only quantified how determinative a genomic region is for cancer presence, or how much coverage a genomic region adds. However, in some configurations, genomic regions and their model coefficients can also indicate cancer type.

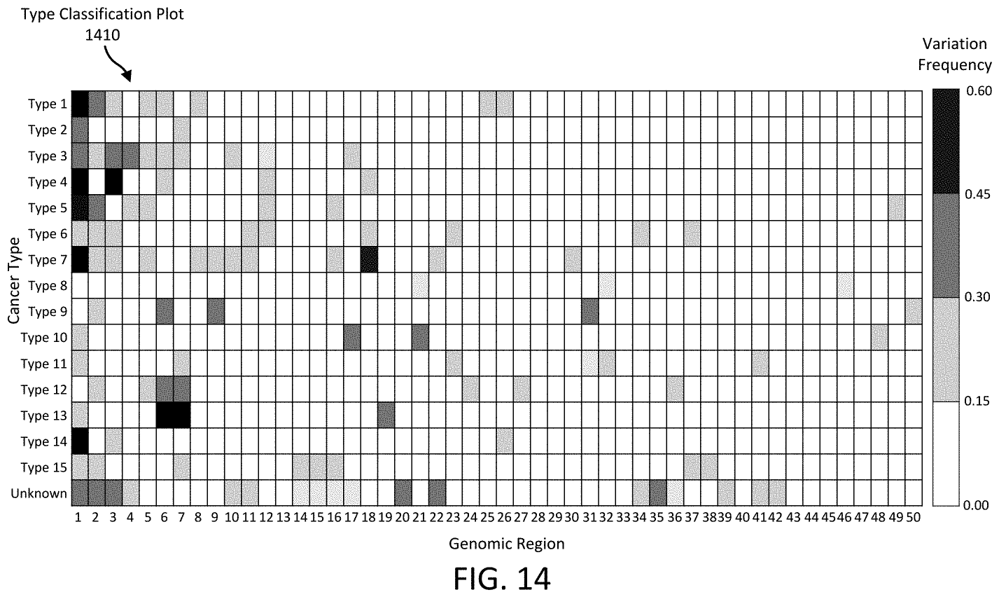

To illustrate, FIG. 14 shows a type classification plot according to some embodiments. A type classification plot illustrates, for a variety of cancer types, a variation frequency for genomic regions. The illustrated type classification plot 1410, shows the frequency of somatic mutations in 50 genomic regions (e.g., 50 selected genes in Tables 11 and 12, above), across fifteen cancer types. The variation frequency ranges from 0.00 to 0.60. The genomic regions are the same, and similarly ranked, as the related indicators in FIGS. 9A-9C. The fifteen cancer types can be, for example, lung, breast, colorectal, pancreatic, esophageal, gastric, hepatobiliary, leukemia, lymphoma, multiple myeloma, bladder, anorectal, head or neck, ovarian, and cervical cancer, respectively. Other cancer types are also possible, though not illustrated.

The classification type plot 1410 illustrates differences in how often a feature variation for a genomic region (e.g., variation in maximum variant allele frequency) occurs in samples having different cancer types. For example, the 1^{st} cancer type is indicated by a feature variation of the 1^{st} genomic region, while the 12^{th} cancer type is rarely indicated by a feature variation for the same genomic region. In another example, the 4^{th} cancer type is indicated by a feature variation of the 3^{rd} genomic region, while the 5^{th} cancer type is rarely indicated by a feature variation for the same genomic region.

For each genomic region, the greater the number of cancer types having a high feature variation, the more likely the genomic region is to indicate cancer presence. That is, genomic regions having high feature variation across several cancer types have higher model coefficients (e.g., sensitivity coefficients). This is illustrated in the type classification plot 1410 as genomic regions on the left side of the plot (i.e., those with higher model coefficients) having an increased density of higher variation frequency across the cancer types over genomic regions on the right side of the plot (i.e., those with lower model coefficients).

In some cases, a feature variation for a genomic region occurs for a single cancer type and no others. For example, a feature variation in the 19^{th} genomic region indicates the 13^{th} cancer type, but no others. This shows that if a panel detects a feature variation for the 19^{th} genomic region, that variation is likely to indicate the 13^{th} cancer type.

Accordingly, some genomic regions can increase the type accuracy of a panel. Type accuracy is a quantification of how accurately a panel determines a cancer type in a sample with a cancer presence. Therefore, to increase type accuracy, the panel generator 250 can apply a cancer type model 276 to determine genomic regions to include in the panel as type indicators.

The cancer type model 276 can be a multinomial logistic regression performed on an indicator set including indicative genomic regions. The panel generator 250 applies the cancer type model 276 to feature values for the indicator set and determines a set of model coefficients for each genomic region (“type coefficients”). The set of type coefficients quantifies the indicativeness of a genomic region for different cancer types. The panel generator 250 then ranks the determined type coefficients for each cancer type, and, subsequently, selects genomic regions from the ranked list for inclusion into the panel as type indicators. The panel generator 250 can select type indicators in ranked order, by some other metric, or not at all.

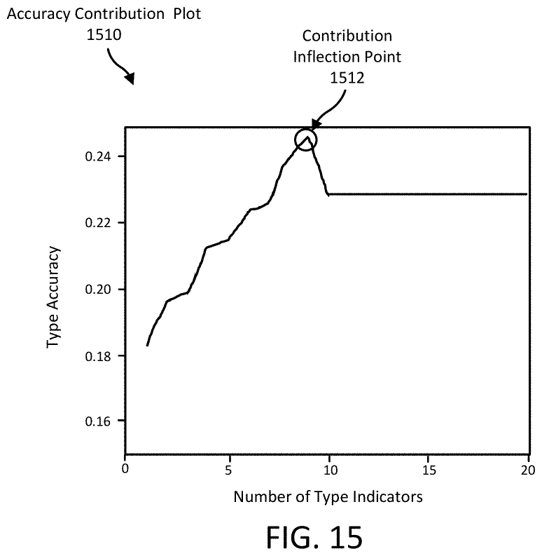

In some embodiments, the panel generator 250 adds type indicators to the panel until subsequent type indicators decrease, or do not contribute to an increase in, the type accuracy of a panel. To illustrate, FIG. 15 shows an accuracy contribution plot for a panel according to some embodiments. In the accuracy contribution plot 1510, the x-axis represents the number of potential type indicators for the panel, and the y-axis illustrates the type accuracy for the panel. The type indicators on the x-axis are selected in ranked order according to their model coefficient.

As shown, adding additional type indicators to the panel increases the type accuracy until a contribution inflection point 1512. At the contribution inflection point 1512, adding type indicators decreases the type accuracy of the panel. In the illustrated example, the contribution inflection point occurs at 9 type indicators, but could be other numbers in other examples. Accordingly, the panel generator 250 can add any combination or all of the 9 additional genomic regions to the panel to increase its type accuracy. For example, the panel generator 250 can select 5 type indicators for the panel. Table 14 indicates the name, size, and position, of the five type indicators selected for the panel.

TABLE 14
Type indicators selected for the panel

| Num. | Gene Name | Size (bp) | Position |
|---|---|---|---|
| 1 | CASP8 | 1,713 | 2q33.1 |
| 2 | EGFR | 3,878 | 7p11.2 |
| 3 | NFE2L2 | 1,818 | 2q31.2 |
| 4 | CDH10 | 2,367 | 5p14.2 |
| 5 | CSMD3 | 11,182 | 8q23.3 |

### X.C Hotspot Indicators

As described above, the panel generator 250 can add any number of genomic regions to a panel to determine a cancer presence. However, in some circumstances, the panel generator 250 can determine that adding one or more portions of a genomic region can determine a cancer presence in a manner similar to adding the full genomic region.

To illustrate, consider a genomic region 1568 bp in length. A feature variation in the genomic region is indicative of a cancer presence. In this example, the feature variation occurs at a 342 bp segment of the genomic region at a particular frequency in the population. If the particular frequency is greater than a threshold frequency (e.g., at least 1% of the population), the panel generator 250 can identify the segment as a hotspot. The panel generator 250 can add the hotspot to a panel as a hotspot indicator (e.g., the 342 bp segment), rather than adding the entire genomic region (e.g., 1568 bp region).

There are several methods to determine hotspot indicators for a panel. In an embodiment, the panel generator 250 can apply a hotspot region model 278 to an indicator set to determine hotspot indicators. The hotspot region model 278 can determine hotspots for any genomic region included in an accessed indicator set. To do so, the panel generator 250 employs the hotspot region model 278 to analyze each genomic region in an indicator set and determine hotspots prone to feature variations. The panel generator 250 can select the hotspots as hotspot indicators for the panel based on one or more criteria. To illustrate, the criteria can include: (i) the hotspot has a feature variation in greater than a threshold percentage of the sample population, (ii) the hotspot is identified when analyzing two or more indicator sets, (iii) the hotspot is identified in a library of segments as possibly indicating cancer presence, (iv) the segment occurs in a genomic region selected for the panel by other models in the classification model 270, (v) the segment does not occur in a genomic region selected for the panel by other models in the classification model 270, and (vi) the hotspot occurs in greater than a threshold number of sequences in the indicator set.

Different criteria selections influence the panel size and detection capability of the panel. For example, a panel generator 250 employing a hotspot region model 278 utilizing the fourth criteria can replace genomic regions with hotspot indicators. Replacing genomic regions with hotspot indicators can reduce the panel size while simultaneously decreasing the detection capability of the panel. Conversely, a panel generator 250 employing a hotspot region model 278 utilizing the fifth criteria can add a significant number of hotspots to the panel. Adding hotspot indicators increases the panel size, and, generally, increases the detection capability of the panel. Many other combinations of criteria are also possible.

In an example, the panel generator 250 selects 36 hotspot indicators for hotspots occurring in greater than 1% of the population that were not previously identified by other models in the classification model 270. Table 15: indicates the name of the genomic region, number of hotspots on that genomic region, and position of 13 hotspot indicators selected for the panel.

TABLE 15
Hotspot indicators selected for the panel

| Num. | Name | Hotspots | Position |
|---|---|---|---|
| 1 | AKT | 1 | 14q32.32 |
| 2 | CDKN2A | 10 | 9p21.2 |
| 3 | DNMT3A | 1 | 2p23.3 |
| 4 | EP300 | 1 | 22q13.2 |
| 5 | ERBB3 | 1 | 12q13.2 |
| 6 | FGFR3 | 2 | 4p16.3 |
| 7 | GNAS | 2 | 20q13.32 |
| 8 | HRAS | 4 | llp15.5 |
| 9 | IDH1 | 2 | 2q32 |
| 10 | IDH2 | 2 | 15q21 |
| 11 | MAPK1 | 1 | 22q11.22 |
| 12 | PTEN | 8 | 10q23.31 |
| 13 | EZH2 | 1 | 7q36.1 |

### X.D Viral Indicators

As described above, the panel generator 250 determines genomic regions indicative of a cancer presence in an indicator set to generate a panel. In some cases, indicator sets include viral genomes that are associated with cancer presence. Accordingly, the panel generator 250 can select genomic regions for viruses associated with cancer presence as viral indicators for a panel. To illustrate, the HPV virus is associated with cervical cancer and is present in a significant fraction of patients having cervical cancer. Accordingly, the panel generator 250 can include viral indicators that increase the detection capability of a panel for cervical cancer.

There are several methods to determine viral indicators for a panel. In an embodiment, the panel generator 250 can apply a viral segment model to determine viral indicators. The viral segment model determines viral indicators from accessed indicator sets. To do so, the panel generator 250 employs the viral segment model to determine a viral coefficient for one or more segments of a viral genome (“viral segments”). The viral coefficient quantifies an association between the viral segment and a cancer presence, and, in some cases, a cancer type. The panel generator 250 then ranks the determined viral coefficients (for classification and/or type), and, subsequently, selects segments from the ranked list for inclusion into the panel as viral indicators. The viral indicators can be selected in ranked order, by some other metric, or not at all. For example, the panel generator 250 can only select viral indicators having a viral coefficient above a threshold value. Additionally, in some cases, the viral segment model can select more than one viral segment per virus for inclusion in the panel. For example, the panel generator 250 can select 10 viral segments of HPV for inclusion into the panel.

Table 16 indicates the name of the virus, the number of viral segments included as viral indicators, and the size of the viral indicators.

TABLE 16
Coverage indicators selected for panel

| Num. | Name | Segments |
|---|---|---|
| 1 | HPV16 | 10 |
| 2 | HPV18 | 10 |
| 3 | EBV | 10 |
| 4 | HBV | 10 |

### XI. Example Panel Generation

As described herein, the panel generator 250 can generate a panel according to several performance metrics, and this section describes several examples of the panel generator 250 generating panels according to a performance metric.

### XI.A Increased Classification Capability

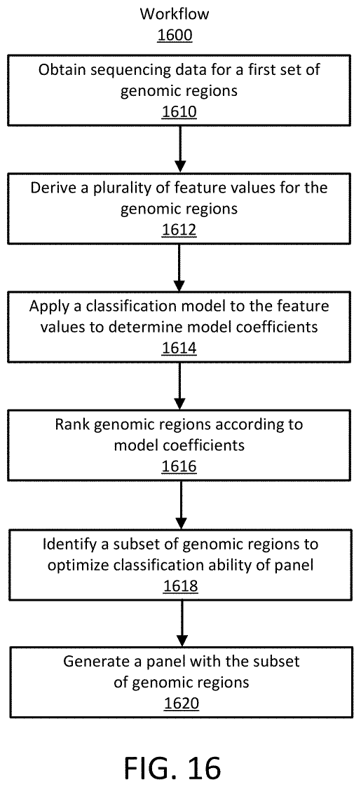

In an example, the performance metric is the classification capability. Accordingly, the panel generator 250 generates a panel for determining a cancer presence. FIG. 16 shows an example workflow for generating a panel for determining a cancer presence according to one embodiment. The workflow 1600 can be executed by the system 200 or another similar system 200. The workflow 400 can include additional or fewer steps, and the steps can be arranged in a different order.

The panel generator 250 obtains 1610 sequencing data (e.g., test sequences) for a first set of genomic regions. The first set of genomic regions can be the CCGA indicator set but could be another set of genomic regions. Each of the genomic regions in the first set is associated with a number of test sequences, and can be associated with cancer-related genes, mutation hotspots, and viral regions.

The panel generator 250 derives 1612 a feature value for each genomic region in the first set. For example, the feature value for each genomic region can be the maxVAF for an SNV of test sequences in the sequencing data associated with that genomic region. Other feature values are also possible. For example, feature values can be an absence or presence of a variant, a mean allele frequency, a total number of small variants, an allele frequency of true variants, etc.

The panel generator 250 employs a classification model 270 that predicts the disease classification ability of the panel based on feature values of genomic regions. The disease classification ability can include classifying, for example, the presence or absence of cancer and/or a type of cancer. The classification ability of the panel, in either case, can be quantified by a performance metric such as, for example, the sensitivity of the panel at a particular specificity.

To predict the disease classification ability, the panel generator 250 applies 1614 the classification model 270 to the feature values to generate a set of model coefficients. Each model coefficient corresponds to a genomic region in the indicator set and quantifies the indicativeness of its corresponding genomic region for disease classification.

The panel generator 250 ranks 1616 the genomic regions according to their model coefficients. For example, the genomic region with the highest model coefficient is ranked first, while the genomic region with the lowest model coefficient is ranked last.

The panel generator 250 identifies 1618 a first subset of the genomic regions based on their rank. For example, the panel generator 250 can identify a subset of the genomic regions that optimizes the disease classification of the panel. The panel generator 250 generates 1620 a panel including the identified first subset of genomic regions.

In some embodiments, the panel generator 250 can access one or more additional sets of indicators and apply the classification model 270 to the additional set of indicators. In doing so, the panel generator 250 can identify one or more additional subsets of genomic regions for inclusion into the panel.

In a first example, the panel generator 250 can access a second indicator set and derive feature values for the genomic regions in the set. When applied to the second indicator set, the classification model 270 determines model coefficients for each genomic region and ranks the genomic regions according to the model coefficients. The classification model 270 can identify a second subset of genomic regions to include in the panel based on their rank. The identified second set of regions can be selected for the panel based on the same, or different, performance metric as the first subset of genomic regions. In a first example, the second set of genomic regions can optimize the coverage of the panel rather than the disease classification ability. In a second example, the selected genomic regions can increase the number of hotspots covered by the panel. In a third example, the selected genomic regions can be associated with a cancer-related virus.

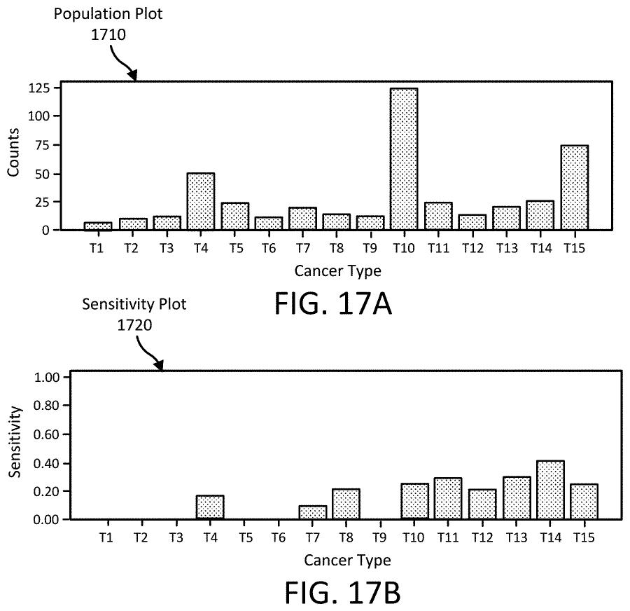

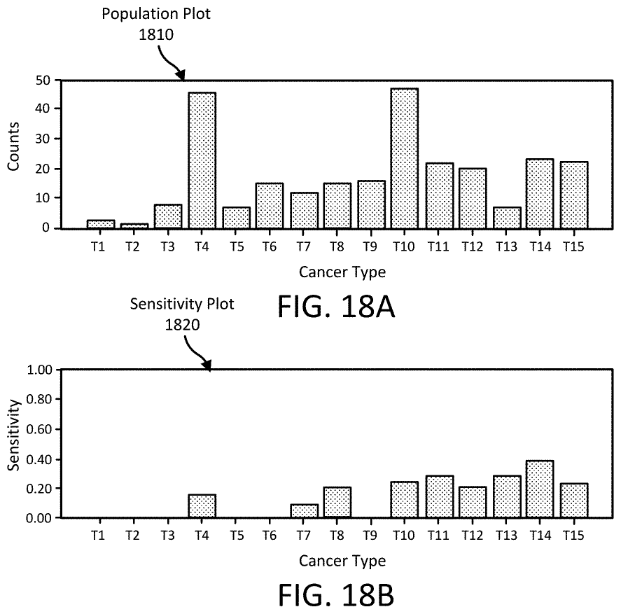

FIGS. 17A-18B illustrate the classification accuracy of a panel generated by the panel generator 250 according to workflow 1600.

FIG. 17A is a population plot for a set of training data according to one embodiment. In a population plot 1710, the x-axis is the type of cancer, and the y-axis is the number of samples having that type of cancer in a training population. In the population plot, the types of cancer are anorectal, bladder, cervical, colorectal, esophageal, gastric, head/neck, hepatobiliary, leukemia, lung, lymphoma, multiple myeloma, ovarian, pancreatic, and breast, respectively.

FIG. 17B is a sensitivity plot according to one example embodiment. In the sensitivity plot 1720, the x-axis is the type of cancer, and the y-axis is the number detection sensitivity of the panel for the training population.

Table 17 illustrates the detection capability of a first panel and a second panel on training data. The first panel is a panel including the related indicators. The second panel is a panel including related indicators, coverage indicator, type indicators, hotspot indicators, and viral indicators. Each entry in the table is the sensitivity at the indicated specificity.

TABLE 17
Detection capability of a panel generated by the panel generator

| Panel | 95% specificity | 98% specificity | 99% specificity |
|---|---|---|---|
| First | 0.6076 | 0.5540 | 0.5299 |
| Second | 0.5912 | 0.5737 | 0.5449 |

FIG. 18A is a population plot for a set of test data according to one embodiment. In a population plot 1810, the x-axis is the type of cancer, and the y-axis is the number of samples having that type of cancer in a test population. In the population plot, the types of cancer are anorectal, bladder, cervical, colorectal, esophageal, gastric, head/neck, hepatobiliary, leukemia, lung, lymphoma, multiple myeloma, ovarian, pancreatic, and breast, respectively.

FIG. 18B is a sensitivity plot according to one example embodiment. In the sensitivity plot 1820, the x-axis is the type of cancer, and the y-axis is the number detection sensitivity of the panel for the test population.

Table 18 illustrates the detection capability of the panel on test data for both a first panel and a second panel. The first panel is a panel including the related indicators. The second panel is a panel including related indicators, coverage indicator, type indicators, hotspot indicators, and viral indicators. Each entry in the table is the sensitivity at the indicated specificity.

TABLE 18
Detection capability of a panel generated by the panel generator

| Panel | 95% specificity | 98% specificity | 99% specificity |
|---|---|---|---|
| First | 0.5092 | 0.4945 | 0.4725 |
| Second | 0.5275 | 0.5091 | 0.4762 |

### XI.B Reduced Panel Size

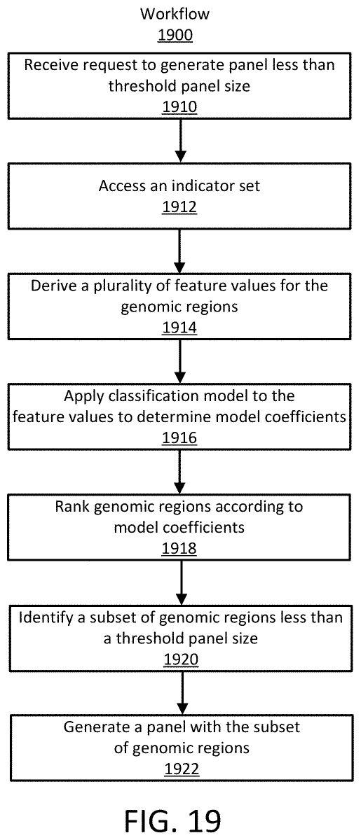

In an example, the performance metric is the panel size. Accordingly, the panel generator 250 generates a panel for determining cancer presence that is less than a threshold panel size. FIG. 19 shows an example workflow for generating a panel less than a threshold panel size according to one embodiment. The workflow 1900 can be executed by the system 200 or another similar system 200. The workflow 1900 can include additional or fewer steps, and the steps can be arranged in a different order.

The system 200 receives 1910 a request to generate a panel that determines a cancer presence in a patient. The request includes a threshold panel size for the panel. In an example, the system 200 receives the request including the threshold panel size from a user of the system 200, but the request can also be received from other sources such as, for example, a connected client system 200, a system 200 administrator, etc. To illustrate, a user of the system 200 transmits a request to the system 200 to generate a panel with a threshold panel size of 400,000 base pairs, but other threshold panel sizes are possible. For example, the threshold panel size can be 10 kb, 35 kb, 70 kb, 150 bk, 300 kb, etc.

The system 200 utilizes a panel generator 250 to determine the one or more genomic regions to include in the panel. The panel generator 250 accesses 1912 an indicator set including sequencing data for genomic regions that can be included the panel. Some example genomic regions included in genomic region databases are described in Tables I-V. In other examples, the sequencing can be accessed, or received, from other sources. For example, the system 200 can receive one or more genomic regions from a user, or the system 200 can determine one or more genomic regions using any of the processes described herein.

The panel generator 250 derives 1914 a feature value for each genomic region in the indicator set, and applies 1916 the classification model 270 to the feature values to determine model coefficients for each genomic region in the indicator set. The panel generator 250 ranks 1918 the determined model coefficients as described above.

The panel generator 250 identifies 1920 a subset of genomic regions for the panel such that the resulting panel has a panel size less than the threshold panel size. To illustrate, continuing the previous example, the threshold panel size for a panel is 16.0 kb. The panel generator 250 iteratively selects genomic regions for the panel, and the corresponding panel size increases based on the size of the selected genomic regions. The panel generator 250 does not select an additional genomic region for the panel if the additional genomic region would cause the resulting panel size to be above the threshold panel size.

The panel generator 250 generates 1922 a panel including the identified first subset of genomic regions. Generating the panel can include transmitting the identified subset of genomic regions to the requestor. For example, the panel generator 250 transmits the panel to the user of the system 200 that requested the panel.

### XI.C Filtering

There are several filtering methods that can improve the detection capability of a panel generated by the panel generator. In a first example, the panel generator can only derive feature values for genomic regions having variants in a threshold number of sequences in the sequencing data. In a second example, the panel generator can duplicate, or remove duplications, of a genomic region from a panel to increase detection capability. In a third example, a system administrator can remove genomic regions from the panel. Finally, the panel generator can remove genomic indicators from the panel based on a genomic region blacklist. The genomic region blacklist can include patented genomic regions, genomic regions known to cause false positives, or any other genomic region that could decrease the detection capability of a panel.

### XII. Generating Probes for an Assay Panel

The panel generator 250 can also employ a probe generator 260 to generate probes for the panel. To do so, the probe generator 260 can input a genomic region selected for the panel and output one or more probes that sequence that genomic region. For example, the probe generator 260 can input a genomic region selected for a panel that is 4.5 kb. The probe generator 260 can output 5 probes to sequence that genomic region (e.g., four 1 kb probes, and one 500 kb probe).

In some examples, the probe generator 260 can normalize probes for a genomic region to a target probe length. In other words, probe generator 260 ensures that all generated probes for a genomic region have the target length. In various embodiments, probe generator 260 can (i) segment a probe to the target length, and/or (ii) augment a probe to the target length when normalizing probes. The probe generator 260 can segment and/or augment a probe any number of times to normalize the probe to the target length.

To illustrate, consider, for example, a panel generated by the probe generator 260 including a first genomic region. The probe generator 260 determines a first probe and a second probe for the first genomic region. The first probe has a size of 2564 nucleobases and the second probe has a size of 112 nucleobases. The target size for probes in the panel is, for example, 120 nucleobases. The probe generator 260 normalizes the first probe by (i) segmenting the first probe into 22 probes, 21 of the probes having 120 nucleobases and 1 of the probes having 44 nucleobases, and (ii) padding the probe having 44 nucleobases to 120 nucleobases. Padding a probe includes appending non-informative nucleobases to the edges of a probe. The probe generator 260 normalizes the second probe by padding the probe to 120 nucleobases.

In some cases, a probe can have a higher probability of incorrectly sequencing a coding region near the edge of the probe. For instance, if a probe includes 120 nucleobases, the, e.g., first ten nucleobases and last ten nucleobases have a higher probability of improperly sequencing the coding regions associated with those nucleobases. Therefore, panel the generator can centralize one or more of probes determined for the panel. Centralizing a probe includes appending non-informative nucleobases to the edges of a probe. To illustrate, consider, for example, a probe for a genomic region including 150 nucleobases. The probe generator 260 centralizes the probe by appending 15 nucleobases to each edge such that the probe includes 180 nucleobases. Other numbers of nucleobases can be appended to the edges of the probe.

In some cases, a probe can improperly sequence a coding region even if it is not near the edge of the probe. As such, the probe generator 260 can tile probes to more accurately sequence a coding region. Tiling a probe includes generating probes in which every nucleobase in a coding regions occurs in at least two probes. Generally, tiled probes are considered adjacent. Adjacent probes are pairs of probes where a fraction of the nucleobases in each probe of the pair are the same. In some examples, the fraction is half, but could be other fractions.

To illustrate consider, for example, a genomic region with a coding region that is sequenced with the following combination of nucleobases: TCGAAACGGTC. The probe generator 260 tiles probes by generating the following probes: (i) [xxTC], (ii) [TCGA], (iii) [GAAA], (iv) [AACG], (v) [CGGT], (vi) [GTCx], and (vii) [Cxxx]. In this example, probes (i) and (ii), (ii) and (iii), (iii) and (iv), etc. are adjacent pairs where half of the probes are the same. With these probes, each nucleobase of the coding region is sequenced two times.

In some embodiments, the probe generator 260 centralize and normalize determined probes. To illustrate, consider, for example, a probe for a genomic region having 330 nucleobases. The target size for a probe is 120 nucleobases. The probe generator 260, in this example, centralizes probes by appending five nucleobases to the edges of each probe. As such, the probe generator 260 centralizes and normalizes the probe by generating three probes of 120 nucleobases. Each of the generated probes have 110 informative nucleobases in the center with 5 non-informative nucleobases on the edges. Other examples of centralizing and normalizing a probe are also possible.

### XIII. Variants Called by the Panel

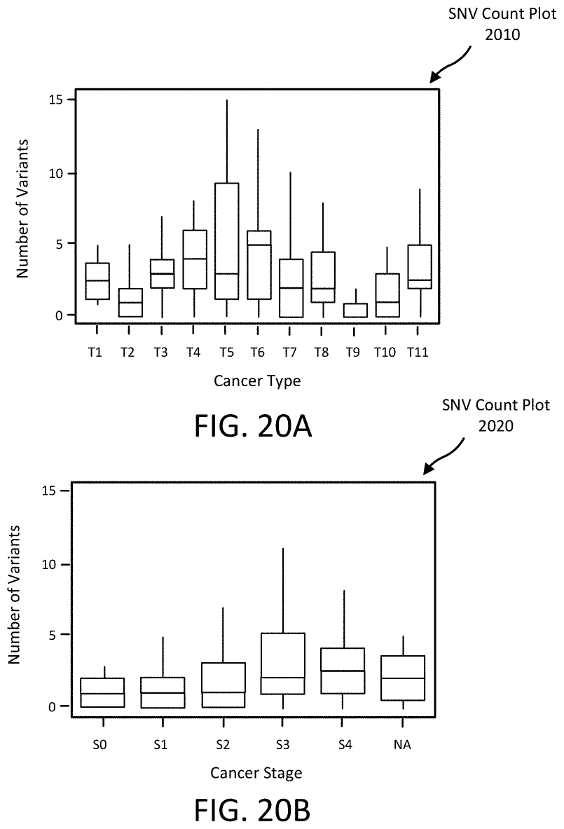

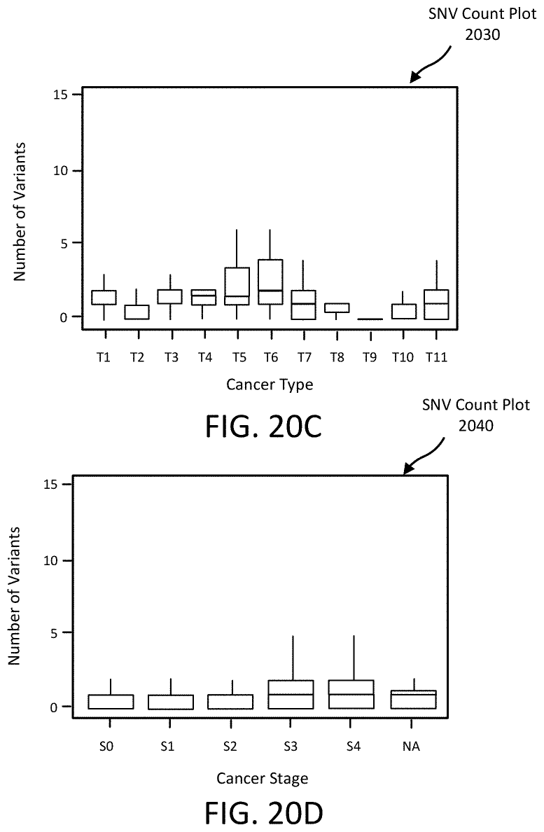

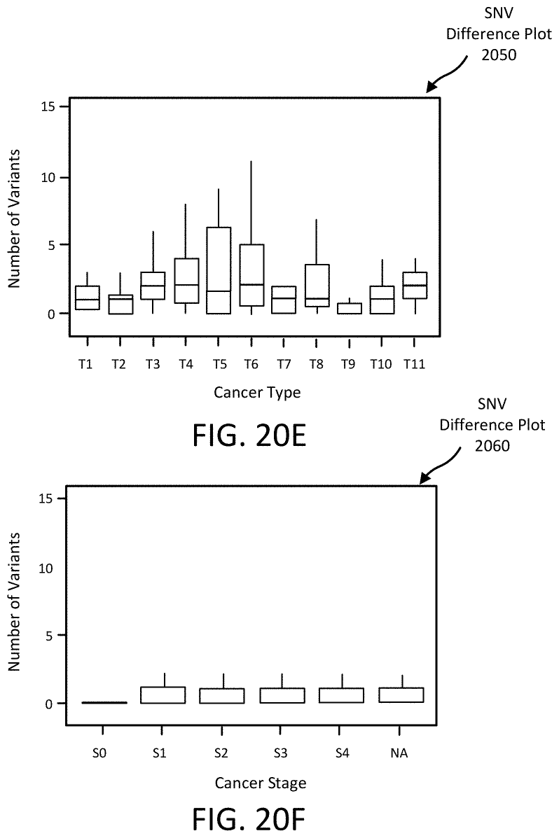

The system 200 can employ a panel generated by the panel generator 250 to call variants. To illustrate, FIGS. 20A-20F give box and whisker plots showing a statistical analysis of the number of variants called by a large set panel, and the number of variants called by a panel generated by the panel generator 250.

FIG. 20A shows an SNV count plot for different cancer types for a large set panel according to one embodiment. In the SNV count plot 2010, the x-axis is the type of cancer, and the y-axis is the number of variants in the sequencing data for that type of cancer. The cancer types can be bladder, breast, colorectal, esophageal, head/neck, lunch, lymphoma, ovarian, renal, and uterine, respectively

FIG. 20B shows an SNV count plot for different cancer stages for a large set panel according to one embodiment. In the SNV count plot 2020, the x-axis is the stage of cancer, and the y-axis is the number of variants in the sequencing data for that stage of cancer.

FIG. 20C shows an SNV count plot for different cancer types for a panel generated using the panel generator according to one embodiment. In the SNV count plot 2030, the x-axis is the type of cancer, and the y-axis is the number of variants in the sequencing data for that type of cancer.

FIG. 20D shows an SNV count plot for different cancer stages for a panel generated using the panel generator according to one embodiment. In the SNV count plot 2040, the x-axis is the stage of cancer, and the y-axis is the number of variants in the sequencing data for that stage of cancer.

FIG. 20E shows an SNV difference plot for different cancer types for a large set panel according to one embodiment. In the SNV difference plot 2050, the x-axis is the type of cancer, and the y-axis is the difference in number of variants in the sequencing data for that type of cancer between the large set panel and the panel generated by the panel generator 250.

FIG. 20F shows an SNV difference plot for different cancer stages for a large set panel according to one embodiment. In the SNV difference plot 2060, the x-axis is the type of cancer, and the y-axis is the difference in number of variants in the sequencing data for that stage of cancer between the large set panel and the panel generated by the panel generator 250.

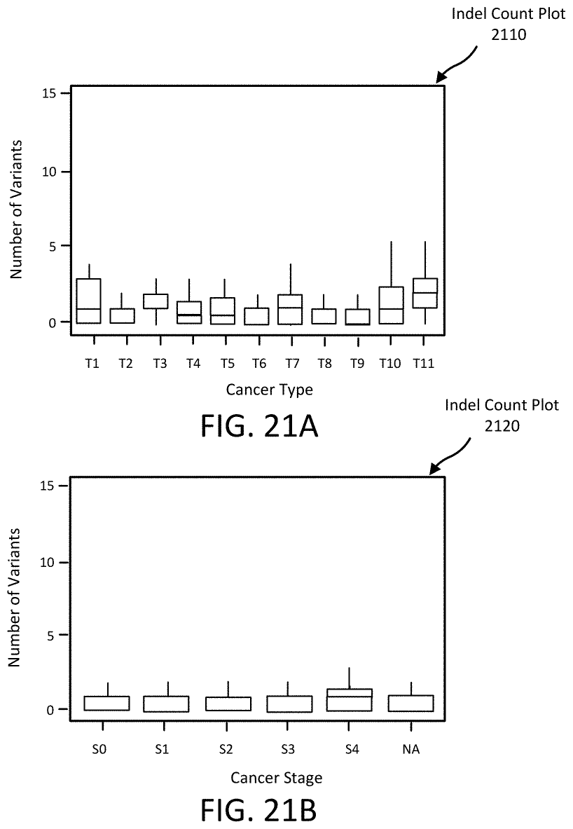

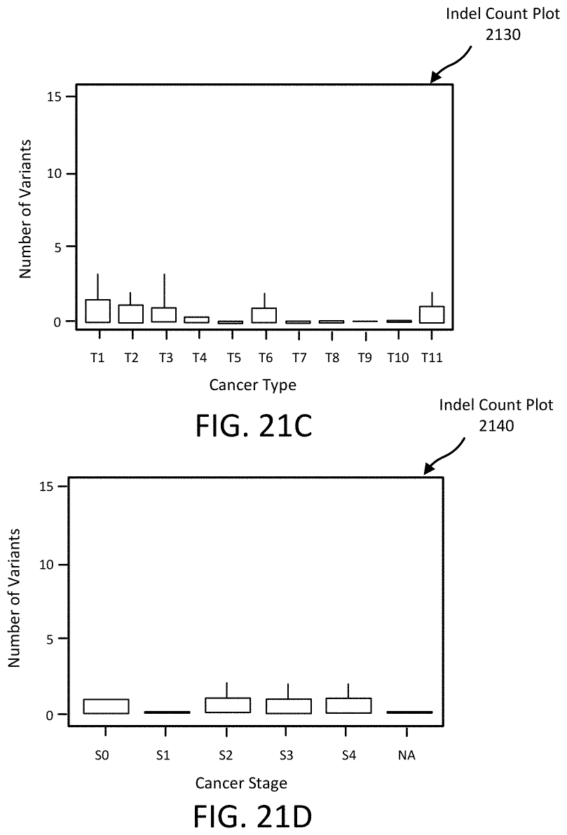

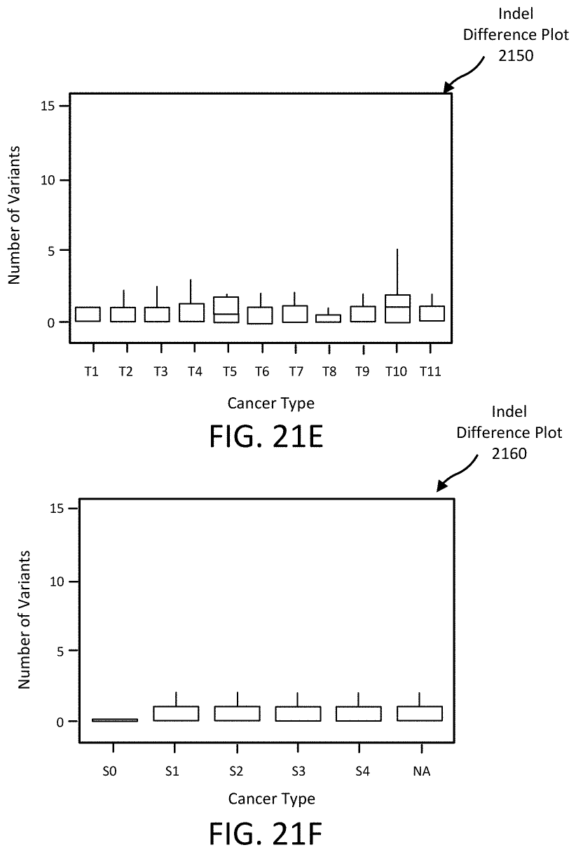

FIG. 21A shows an indel count plot for different cancer types for a large set panel according to one embodiment. In the indel count plot 2110, the x-axis is the type of cancer, and the y-axis is the number of variants in the sequencing data for that type of cancer. The cancer types can be bladder, breast, colorectal, esophageal, head/neck, lunch, lymphoma, ovarian, renal, and uterine, respectively

FIG. 21B shows an indel count plot for different cancer stages for a large set panel according to one embodiment. In the indel count plot 2121, the x-axis is the stage of cancer, and the y-axis is the number of variants in the sequencing data for that stage of cancer.

FIG. 21C shows an indel count plot for different cancer types for a panel generated using the panel generator according to one embodiment. In the indel count plot 2130, the x-axis is the type of cancer, and the y-axis is the number of variants in the sequencing data for that type of cancer.

FIG. 21D shows an indel count plot for different cancer stages for a panel generated using the panel generator according to one embodiment. In the indel count plot 2140, the x-axis is the stage of cancer, and the y-axis is the number of variants in the sequencing data for that stage of cancer.

FIG. 21E shows an indel difference plot for different cancer types for a large set panel according to one embodiment. In the indel difference plot 2150, the x-axis is the type of cancer, and the y-axis is the difference in number of variants in the sequencing data for that type of cancer between the large set panel and the panel generated by the panel generator 250.

FIG. 21F shows an indel difference plot for different cancer stages for a large set panel according to one embodiment. In the indel difference plot 2160, the x-axis is the type of cancer, and the y-axis is the difference in number of variants in the sequencing data for that stage of cancer between the large set panel and the panel generated by the panel generator 250.

### XIV. Additional Considerations

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules can be embodied in software, firmware, hardware, or any combinations thereof.

Any of the steps, operations, or processes described herein can be performed or implemented with one or more hardware or software modules, alone or in combination with other devices. In one embodiment, a software module is implemented with a computer program product including a computer-readable non-transitory medium containing computer program code, which can be executed by a computer processor for performing any or all of the steps, operations, or processes described.

Embodiments of the invention can also relate to a product that is produced by a computing process described herein. Such a product can include information resulting from a computing process, where the information is stored on a non-transitory, tangible computer readable storage medium and can include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

1-35. (canceled)

36. A method for identifying a disease state, the method comprising:
detecting, from a cell-free nucleic acid sample obtained from a subject, a somatic mutation in at least one gene in a set of genes, wherein the set of genes comprises:
three or more genes from a first group comprising: KRAS, TP53, ERBB2, EPHB1, NRAS, ACVR1B, TP63, KEAP1, CDK12, KMT2D, DICER1, TET2, LATS2, ETV5, GRIN2A, EPHA7, ASXL2, RET, CHD2, RB1, CDH1, PDGFRA, BRCA2, TFRC, ALK, KDM5A, SMAD4, ATR, NOTCH1, NRG1, CTNNB1, KMT2C, SNCAIP, MTOR, PIK3CA, SF3B1, NBN, LRP1B, TNFRSF14, ARID1A, INPP4A, ETS1, KAT6A, FBXW7, MGA, MYD88, CBL, BRAF, CREBBP, and APC; and
one or more genes from a second group of genes associated with viral hotspots, the second group of genes comprising: HPV16, HPV18, EBV, and HBV; and
determining the disease state based on the detected somatic mutation.

37. The method of claim 36, wherein the set of genes comprises five or more genes in the first group.

38. The method of claim 36, wherein the set of genes comprise ten or more genes in the first group.

39. The method of claim 36, wherein the set of genes comprises KRAS, TP53, ERBB2, EPHB1, NRAS, ACVR1B, TP63, and KEAP1.

40. The method of claim 39, wherein the set of genes further includes one or more of CDK12, KMT2D, DICER1, TET2, LAT52, ETV5, GRIN2A, EPHA7, ASXL2, and RET.

41. The method of claim 36, wherein the set of genes comprises TP53, NRAS, KMT2D, TET2, KMT2C, SF3B1, LRP1B.

42. The method of claim 41, wherein the set of genes further includes one or more of MYD88, CBL, BRAF, CREBBP, AND APC.

43. The method claim 36, wherein detecting the somatic mutation comprises detecting a single nucleotide variant.

44. The method of claim 43, wherein detecting the somatic mutation further comprises detecting an indel.

45. The method of claim 36, wherein the set of genes further comprises one or more genes from a third group of genes associated with hotspots for SNVs and indels, the third group of genes consisting of: AKT1, ERBB3, IDH1, PTEN, ARAF, EZH2, IDH2, PTPRD, CD79A, FGFR3, MAP3K1, RHOA, CDKN2A, GATA3, MAPK1, RNF43, DNMT3A, GNAS, MSH2, SPTA1, EP300, HRAS, PREX2 and TERT.

46. (canceled)

47. The method of claim 36, further comprising:
developing a therapy, prognosis, or diagnosis in accordance with the at least one gene and the somatic mutation detected at the at least one gene.

48-50. (canceled)

51. A cancer assay panel, comprising:
one or more genes selected from a first group of genes associated with high signal cancers or liquid cancers;
one or more genes selected from a second group of genes associated with hotspots for single nucleotide variants (SNVs) or indels; and
one or more genes selected from a third group of genes associated with viral hotspots, the third group of genes comprising: HPV16, HPV18, EBV, and HBV.

52. The panel of claim 51, wherein the first group of genes comprises: KRAS, TP53, ERBB2, EPHB1, NRAS, ACVR1B, TP63, KEAP1, CDK12, KMT2D, DICER1, TET2, LATS2, ETV5, GRIN2A, EPHA7, ASXL2, RET, CHD2, RB1, CDH1, PDGFRA, BRCA2, TFRC, ALK, KDMSA, SMAD4, ATR, NOTCH1, NRG1, CTNNB1, KMT2C, SNCAIP, MTOR, PIK3CA, SF3B1, NBN, LRP1B, TNFRSF14, ARID1A, INPP4A, ETS1, KAT6A, FBXW7, MGA, MYD88, CBL, BRAF, CREBBP, and APC.

53. The panel of claim 51, wherein the second group of genes comprises a set of genes associated with hotspots for SNVs, the set of genes comprising: AKT1, CDKN2A, DNMT3A, EP300, ERBB3, FGFR3, GNAS, HRAS, IDH1, IDH2, MAP3K1, MAPK1, PREX2, PTEN, PTPRD, RHOA, SPTA1, TERT, and EZH2.

54. The panel of claim 51, wherein the second group of genes comprises a set of genes associated with indels, the set of genes comprising: ARAF, CD79A, GATA3, MSH2, PTEN, and RNF43.

55. (canceled)

56. The panel of claim 51, wherein the assay panel detects a presence or absence of cancer in a subject or detects a type of cancer in the subject.

57. (canceled)

58. A cancer detection panel for determining a presence or absence of cancer in a patient, wherein the cancer detection panel is manufactured by a process comprising:
identifying a plurality of genomic regions, each genomic region associated with a likelihood that a variation in a feature of the genomic region is indicative of cancer, and each genomic region having a kilobase size;
applying a classifier model to the plurality of genomic regions, the classifier model configured to:
determine a sensitivity score for each one of the genomic regions, the sensitivity score quantifying a contribution to a detection sensitivity of the cancer detection panel, the detection sensitivity quantifying the likelihood that variations of the features in the set of genomic regions included in the cancer detection panel are indicative of cancer,
rank the plurality of genomic regions according to their sensitivity score, and
select, based on their rank, one or more of the genomic regions as the set of genomic regions for the cancer detection panel, the sum of the kilobase sizes for set of genomic regions in the detection panel less than an aggregate kilobase size associated with the cancer detection panel; and
generating the cancer detection panel based on the selected one or more genomic regions.

59-60. (canceled)

61. The cancer detection panel of claim 58, wherein the feature of the genomic region is a single nucleotide variant.

62. The cancer detection panel of claim 61, wherein the variation of the feature that is indicative of cancer is a maximum variant allele frequency for the single nucleotide variant of the genomic region.

63. The cancer detection panel of claim 58, wherein one or more of the genomic regions indicates a virus associated with cancer.

64. The cancer detection panel of claim 63, where the virus is any of HPV16, HPV18, EBV, and HBV.

65. The cancer detection panel of claim 58, wherein one or more of the genomic regions are associated with solid cancers.

66. (canceled)

67. The cancer detection panel of claim 58, wherein one or more of the genomic regions are associated with liquid cancers.

68. (canceled)

69. The cancer detection panel of claim 58, wherein one or more of the genomic regions indicates a cancer hotspot.

70. (canceled)

71. The cancer detection panel of claim 58, wherein one or more of the genomic regions are associated with a specific type of cancer.

72-74. (canceled)

75. The cancer detection panel of claim 58, wherein ranking the indicators further comprises:
ranking the genomic regions based on a type of cancer that the detection panel is designed to detect.

76-142. (canceled)
